SEQUENCE LISTING (16 sequences)

SEQ ID NO: 1 - DNA, 29 bp, Human Papillomavirus
gatggtgatatggtagatacaggatttgg

SEQ ID NO: 2 - DNA, 25 bp, Human Papillomavirus
ggcgatatggttgatacaggctttg

SEQ ID NO: 3 - DNA, 27 bp, Human Papillomavirus
gcacaactatttaataagccatattgg

SEQ ID NO: 4 - DNA, 35 bp, Human Papillomavirus
ttcttcttacgaagggaacaactgtttgttagaca

SEQ ID NO: 5 - DNA, 24 bp, Human Papillomavirus
tgatatggttcatacaggatttgg

SEQ ID NO: 6 - DNA, 33 bp, Human Papillomavirus
tgtacctgctattggggaacactgggctaaggg

SEQ ID NO: 7 - DNA, 23 bp, Human Papillomavirus
  misc_feature (15)..(15): n is a, c, g, or t
gaggtgggccggggncarccnyt

SEQ ID NO: 8 - DNA, 29 bp, Human Papillomavirus
gcgtcagaggttaccatagagccactagg

SEQ ID NO: 9 - DNA, 24 bp, Human Papillomavirus
agaagtaaccatagagccactagg

SEQ ID NO: 10 - DNA, 24 bp, Human Papillomavirus
aataaactgtaaatcatattcctc

SEQ ID NO: 11 - DNA, 35 bp, Human Papillomavirus
cataattgaaacataaactgtaaatcatattcctc

SEQ ID NO: 12 - DNA, 33 bp, Human Papillomavirus
taattgggaatcagaagtaaccatagagccact

SEQ ID NO: 13 - DNA, 28 bp, Human Papillomavirus
  misc_feature (21)..(21): n is a, c, g, or t
aagccggtgtcgaccatrtcnccrtcyt

SEQ ID NO: 14 - DNA, 28 bp, Human Papillomavirus
  misc_feature (24)..(24): n is a, c, g, or t
ccgaagccggtgtcgaycatrtcnccrt

SEQ ID NO: 15 - DNA, 20 bp, Human Papillomavirus
cgtccmarrggawactgatc

SEQ ID NO: 16 - DNA, 20 bp, Human Papillomavirus
gcmcagggwcataayaatgg

(19) United States
(12) Patent Application Publication
(10) Pub. No.: US 2004/0248085 A1
(43) Pub. Date: Dec. 9, 2004

(54) General primers and process for detecting diverse genotypes of human papillomavirus by PCR

(76) Inventors: Sang-Wha Lee, Yongin-si (KR); Yeon-Soo Kim, Seoul (KR); Kang-Yeol Yu, Jeonju-si (KR); Seung-Jo Kim, Seoul (KR); Kwang-Yul Cha, Seoul (KR); Jung-Jae Ko, Yongin-si (KR)

Correspondence Address: BAKER & DANIELS, 300 NORTH MERIDIAN STREET, SUITE 2700, INDIANAPOLIS, IN, 46204-1782, US

(21) Appl. No.: 10/720,424

(22) Filed: Nov. 24, 2003

(30) Foreign Application Priority Data
Nov. 29, 2002 (KR) 2002-75370
Jul. 31, 2003 (KR) 2003-53147

(51) Int. Cl. C12Q 1/70; C07H 21/04; C12P 19/34

(52) U.S. Cl. 435/5; 435/91.2; 536/24.1

(57) ABSTRACT

The presence of diverse human Papillomavirus genotypes in a sample can be detected by 1 cycle of polymerase chain reaction using primers. The primers used in the method are general primers which can bind complementarily to specific nucleic acid sequences of all HPV genotypes related to cervical carcinoma and its pre-stage lesions and can be used to amplify cervical-neoplasia related HPV genotypes including oncogenic high-risk groups and low-risk groups.

This application claims the benefit of Korean Patent Application No. 2002-0075370, filed on Nov. 29, 2002 in Korea and Korean Patent Application No. 2003-0053147, filed on Jul. 31, 2003 in Korea, which are hereby incorporated by reference for all purposes as if fully set forth herein.

### BACKGROUND OF THE INVENTION

1. Field of the Invention

The present invention relates to PCR primers for detecting and/or diagnosing the presence or absence of Human papillomavirus (HPV) in a sample and a method for detecting and/or analyzing the presence or absence of HPV in a sample using the primers and more particularly, to PCR primers consisting of oligonucleotides which can bind complementarily to various types of HPV nucleic acid and thereby obtaining amplification products and a method for detecting and/or analyzing the presence or absence of various types of HPV in a sample using the primers.

2. Discussion of the Related Art

Human papillomavirus (HPV) is related to varieties of tumors including cervical cancer and approximately 120 different HPV genotypes are discovered to date. HPV is a circular double-stranded DNA virus with its genome size of about 8 kb. There are E1 to E7 genes concerning an early step and L1 and L2 genes concerning a late step after infecting a host cell in the HPV genome. Especially, each of proteins E6 and E7, which are oncogenic proteins being expressed after HPV infects an epithelial cell of a host, binds specifically to p53 and pRB, which are oncogenic suppression protein of the host cell and thereby eliminating the oncogenic suppression function of these host proteins. Accordingly, the E6 and E7 proteins of HPV cause the infected host cell to transform and develop to tumor formation.

To date, more than 120 different HPV genotypes are discovered according to a sequence difference of ORFs (open reading frames) of genes E6, E7 and L1. It is defined as new genotypes, new subtypes and new variants, in case the nucleotide sequence difference of the ORFs is more than 10%, 2 to 10%, and less than 2% respectively. Particularly, HPV types 2,3,6,7, 10, 11, 13, 16, 18,26,30,31,32, 33,34,35,39,40,42,43, 44,45,51,52,53,54, 55, 56, 57, 58, 59, 61, 66, 67, 68, 69, and 70 are known as cervical-cancer causing primary viruses.

According to WHO, there happened about 450,000 of new cervical cancer patients each year worldwide, which is the first in women cancers but a breast cancer. Especially, the cervical cancer is most ordinary women cancer and it cause about 300,000 to die in developing country.

After HPV infects cervical epithelial base cells, they develop premonitory syndromes such as low squamous intraepithelial lesion (LSIL), high squamous intraepithelial lesion (HSIL) and intraepithelial neoplasia and finally to an invasive carcinoma. Since there are premonitory stages prior to developing a carcinoma, it is possible to diagnose these premonitory syndromes in early stage and thereby preventing a cervical cancer. Israel, which has established an early medical examination for cervical cancer and its pre-stage disorders, has a frequency of cervical cancer 3.8 persons per hundred thousand capita. On the other hand, developing countries such as Columbia have a frequency of cervical cancer 48.2 persons per hundred thousand capita.

Conventionally, cell smear testing based on a cytological morphology of cervical cell smear for selecting primarily cervical cancer and its pre-stage lesion has been practiced since 1940's. However, the cytological testing such as Pap smear testing, is not subjective and shows a high false-negative of 30˜40%.

Also, HPV hybrid capture II, which detects a liquid state complementary hybridizing compound between DNA of cervical cancer smear and RNA probe by an antibody enzyme colorization, has been used for an auxiliary selection or for a follow-up of cervical cancer and its pre-stage lesions. However, this testing has not been used widely since it is highly expensive. Accordingly, there still remains a need to develop a method for diagnosing generally cervical carcinoma and its pre-stage disorders at early stage.

Therefore, a great deal of research has been done to detect general HPV types at early stage by using PCR method, which can amplify DNA fragments of specific nucleotide sequence simply and fast, in order to detect multiple HPV types in a sample.

PCR (polymerase chain reaction) method, which is a technology to detect a target nucleic acid sequence in a specific organism by adding nucleic acid fragments (oligo primers) complementary to the target sequence and amplifying a small entity of the target sequence by repeating denaturation, annealing and polymerization according to temperature alterations, is disclosed in U.S. Pat. No. 4,683,195 and 4,683, 202, which are incorporated herein by reference. By using PCR method, it is possible to detect whether a sample DNA is infected by HPV or not easily.

In view of practically clinical examination, the PCR method has an advantage over a cytological testing such as Pap smear since it is subjective and can test a HPV infection on a large scale. Also, PCR method has an advantage over a cytological testing or a liquid state hybrid capture method in testing cost, processing time, detection sensitivity, detection specificity and the like.

Accordingly, there are a lot of patents for detecting and/or diagnosing HPV types in a sample using the PCR method. For example, U.S. Pat. No. 5,484,699 (Bouma Stanley et al.), which is incorporated herein by reference, discloses nucleotides for amplifying DAN fragments of high-risk HPV types specifically concerned with cervical carcinoma and a method for detecting high-risk HPV types using the nucleotides. However, U.S. Pat. No. 5,484,699 discloses nucleotides to produce an amplification product to a specific HPV types DNA and therefore it cannot detect a non high-risk HPV types.

Also, U.S. Pat. No. 6,352,825, which is incorporated herein by reference, disclosed primers to amplify nucleic acids of comparatively diverse HPV types. But, the primers amplifies only nucleic acids of high-risk groups such as HPV types 16, 18, 31, 33, 35, 39, 45, 51, 52, 54, 55, 56, 57, 58. Accordingly, the primers also cannot detect low-risk HPV types or other types.

Presently, there are used general primers for diagnosing HPV infection or not in clinical examination worldwide. For example, MY09/MY11 primer sets are used mainly in North America, South America and Asia, GP5+/GP6+primer sets which are used widely in Europe and other newly developed primer sets such as FAP59/FAP64, PGMY09/PGMY11 are widely used.

However, those primer sets widely used worldwide have disadvantages since they can detect limited HPV types comparing with diverse HPV types classified into more than 120 types and shows a lack of sensitivity according to HPV types. Accordingly, use of those general primer sets in a clinical field is limited though they have other values and needs.

Therefore, there still remains a need to develop general primers not only to produce amplifying products to DNA of scores of diverse oncogenic HPV types and thereby detecting the oncogenic HPV types in a sample but also to select cervical carcinoma and its pre-stage lesions at early stage by improving sensitivity according to HPV types.

### SUMMARY OF THE INVENTION

Accordingly, the present invention is directed to a general primer to detect simultaneously DNA of diverse classified HPV types that substantially obviates one or more of problems due to limitations and disadvantages of the related art.

An advantage of the present invention is to provide a general primer that can bind complementarily to oncogenic HPV types that are related to a cervical carcinoma or its pre-stage lesions and detect a presence or absence of the oncogenic HPV types in a sample.

Another advantage of the present invention is to provide a method for detects

Additional features and advantages of the invention will be set forth in the description which follows, and in part will be apparent from the description, or may be learned by practice of the invention. These and other advantages of the invention will be realized and attained by the structure particularly pointed out in the written description and claims hereof as well as the appended drawings.

To achieve these and other advantages and in accordance with the purpose of the present invention, as embodied and broadly described, a general primer for detecting HPV genotypes, wherein the primer is an oligonucleotide selected from SEQ ID NO: 1 to SEQ ID NO: 14, is provided.

In another aspect, the present invention provides a general primer pair which can be used in a nucleic acid amplification process for amplifying of HPV genotypes, wherein a first primer is an oligonucleotide selected from SEQ ID NO: 1 to SEQ ID NO: 7 and a second primer is an oligonucleotide selected from SEQ ID NO: 8 to SEQ ID NO: 14.

In another aspect, the present invention a method for amplifying DNA of Human Papillomavirus genotypes comprises performing a nucleic acid amplification process using at least one primer pair selected from the general primer pairs.

In another aspect, the present invention provides a method of analyzing a sample for the presence therein of Human Papillomavirus genotypes which comprises the steps of: (a) amplifying DNA of a Human Papillomavirus in the sample by means of a nucleic acid amplification process using at 1 east one primer pair selected from the general primer pairs; and (b) detecting an amplification product wherein the occurrence of the amplification product indicates presence of Human Papillomavirus genotypes in the sample.

Further, in another aspect, the present invention provides a PCR kit that comprises the general primer pairs, polymerase for amplifying nucleic acids, buffer solution, and 4 kinds of nucleoside.

It is to be understood that both the foregoing general description and the following detailed description are exemplary and explanatory and are intended to provide further explanation of the invention as claimed.

### BRIEF DESCRIPTION OF THE DRAWING

The accompanying drawings, which are included to provide a further understanding of the invention and are incorporated in and constitute a part of this specification, illustrate embodiments of the invention and together with the description serve to explain the principles of the invention.

In the drawings:

FIGS. 1A to 1C are respectively gel electrophoresis photographs showing the PCR results when primer pairs, which are produced according to one embodiment of the present invention, were used to amplify HPV-16 infected cell-line Caski, HPV-18 infected cell-line HeLa and HPV non-infected cell-line K562;

FIGS. 2A to 2h are respectively gel electrophoresis photographs showing the PCR results when primer pairs, which are produced according to another embodiment of the present invention, were used to amplify HPV-16 infected cell-line Caski, HPV-18 infected cell-line HeLa and HPV non-infected cell-line K562;

FIGS. 3A to 3C are respectively gel electrophoresis photographs showing the results when 3 primer pairs, which are produced according to one embodiment of the present invention, were used to amplify selected HPV DNA containing plasmids;

FIGS. 4A to 4G are respectively gel electrophoresis photographs showing the result when 7 primer pairs, which are produced according to another embodiment of the present invention, were used to amplify selected HPV DNA containing plasmids;

FIG. 5 is a gel electrophoresis showing the PCR result when a primer pair, which is produced according to another embodiment of the present invention, was used to amplify selected HPV DNA containing plasmids;

FIGS. 6A to 6T are respectively gel electrophoresis showing the PCR results when 3 primer pairs, which are produced according to one embodiment of the present invention, and conventionally used primer pair for amplifying HPV DNA were used to amplify DNA obtained from cervical-cancer related subjects by conventional examinations;

FIGS. 7A to 7j are respectively gel electrophoresis showing the PCR results when 3 primer pairs, which are produced according to one embodiment of the present invention, and conventionally used primer pair for amplifying HPV DNA were used to amplify DNA obtained from cervical-cancer non-related subjects by conventional examination;

FIGS. 8A to 8T are respectively gel electrophoresis showing the PCR results when 7 primer pairs, which are produced according to another embodiment of the present invention, were used to amplify DNA obtained from cervical-cancer related subjects by conventional examinations; and

FIGS. 9A to 9j are respectively gel electrophoresis showing the PCR results when 7 primer pairs, which are produced according to another embodiment of the present invention, were used to amplify DNA obtained from cervical-cancer non-related subjects by conventional examination.

### DETAILED DESCRIPTION OF THE ILLUSTRATED EMBODIMENTS

As mentioned above, the conventional oligonucleotide general primer pairs, which can hybridize various types HPV DNA by PCR, detect only a relatively limited types to existing HPV types and shows a lack of sensitivity according to HPV types. The present invention designs primer pairs which can hybridize more HPV types than the conventional general PCR primer pairs and thereby detecting more various HPV types by carrying out a nucleic acid amplification process using the primer pairs of the invention.

First of all, the present invention designs a representative general PCR primers using computer program based upon full-length nucleotide sequences of 72 HPV types. the designed PCR primers of the present invention c an bind complementarily specific nucleic acid sequences of cervical-cancer related all types HPV including not only high-risk groups also low-risk groups and form amplification product to the HPV types.

The designed primers according to the present invention is oligonucleotides which have a limited sequence length of about 30 bps and produce a amplification product of 200˜400 bps. In the limited conditions, the inventors designed 14 oligonucleotide sequences. Among the 14 oligonucleotides, 7 nucleotides are designed for using as 5′ primers (forward primers) and other 7 nucleotides are designed for using as 3′ primers (reverse primers).

Continuously, the inventor of the present invention selected at least one nucleotide among the designed 5′ primers and at least one nucleotide among the designed 3′ primers to synthesize primer pairs and certified their amplification results.

Any one of oligonucleotide sequence among the designated 5′ primers and the 3′ primers of the present invention can bind complementarily to nucleic acid sequences of various HPV types including cervical-cancer related HPV types. However, especially primer pairs, which comprise a specific oligonucleotide sequence selected from oligonucleotides consisting the 5′ primers and a specific oligonucleotide sequence selected from oligonucleotides consisting the 3′ primers, can amplify nucleic acids of more various HPV types.

Through the above-mentioned method, selected 11 primer pairs were used to amplify nucleic acids of more various HPV types. The selected Primers are synthesized by using automated nucleic acid sequencer (Expedite™ 8909 Synthesizer, ABI Corporation). Those synthesized primers are used to amplify DNAs of representative HPV-infected cell-lines in PCR process and they are certified to produce amplification products by electrophoresis.

Also, the synthesized 11 primer pairs were used to amplify various plasmids containing a HPV DNA in PCR and electrophoresis process to verify amplification products according to their expected amplification experiments.

Further, it is possible to diagnose whether HPV types infect a sample obtained from cervical smear of subjects or not by carrying out PCR process using the synthesized 11 primer pairs to amplify the sample and by certifying a presence or absence of amplification products.

To use the synthesized primer pairs in clinical examinations, the inventors examined samples obtained from cervical smears of subjects through clinically pathological testing such as colopscopy and classified the subjects into abnormal subjects who are related to cervical carcinoma and normal subjects who are not related to cervical carcinoma. Then, some of abnormal subjects and normal subjects are tested to certify HPV infection more specifically by applying hybrid capture II (DIGENE Corp.) and DNA chip test (BIOMDERAB Co. Ltd.).

Consequently, the general primers of the present invention and already-known representative general primer pair MY09/MY 11 were used to amplify each of the sample DNAs obtained from the abnormal subjects or normal subjects. The present inventors certified that the general primer pairs of the present invention can produce amplification products which is identical to the results of the cytological test, hybrid capture II and DNA chip test and that can be used to diagnose whether samples are infected by HPV or not correctly and early. That is, the primer pairs of the present invention produced amplification products to the DNA samples obtained from subjects who are examined as related to cervical carcinoma, but did not produce amplification products to the DNA samples obtained from subjects who are examined as not related to cervical carcinoma.

The following examples are illustrative for describing the present invention in more detail. However, the present invention is not limited to the following examples.

### EXAMPLES

### Example 1

### PCR Primer Design and Synthesis

(1) PCR Primer Design

Nucleotide sequences as PCR general primers that can bind complementarily to various HPV types and thereby amplifying the HPV types DNA totally was designed according to the object of the present invention.

First, full-length nucleic acid sequences of 72 HPV types such as HPV 1a, 2a, 3,4,5, 6b, 7,8,9, 10, 11, 12, 13, 15, 16, 16r, 17, 18, 19, 20, 21, 22, 23, 24, 25, 26, 27, 28, 29, 30, 31, 32, 33, 34, 35h, 36, 37, 38, 39, 40, 41, 42, 44, 45, 47, 48, 49, 50, 51, 52, 53, 54, 55, 56, 57, 58, 59, 60, 61, 63, 65, 66, 67, 68, 70, 72, 73, 74, 75, 76, 77, and 80 were derived from NCBI (National Center for Biotechnology Information) and National HPV database of Los Alamos of United States.

DNA sequence homology studies were performed with the derived DNA sequences using Clustal W computer program (DNSSTAR, MegAlign™ 5, DNASTAR Inc.) for pairwise alignment, multiple sequence alignment and phylogenetic tree to screen representative base sequences of groups. Then, PCR general primers based on the screened base sequences were designed using another computer program (primer premier 6, PREMIER Biosoft International Co.). The primers were set as a nucleotide sequence of about 27±3 bps or about 31±2 bps and their amplification products of about 200˜500 bps and 30 PCR general primer pairs were designed.

(2) Virtual Amplification and Synthesis of PCR Primer

Each of the designed 30 primer pairs was directed to amplify virtually 72 HPV types that are derived in the above primer-designing steps using another computer program (Amplify 1.2, University of Wisconsin). The designed PCR general primer pairs were used to certify oligonucleotide sequences to amplify DNAs of various HPV types, especially cervical-carcinoma related 36 HPV types such as HPV 2a, 3, 6b, 7, 10, 11, 13, 16, 18, 26, 30, 31, 32, 33, 34, 35, 39, 40, 42, 44, 45, 51, 52, 53, 54, 55, 56, 57, 58, 59, 61, 66, 67, 68, 70, 73, and 74. Some of the certified oligonucleotide sequences are used as 5′ primers (forward primers) and others are used as 3′ primers (reverse primers).

The selected 5′ primers and 3′ primers were randomly combined to perform a virtual amplification. Then, three primer pairs to amplify DNA of various HPV types were certified. Hereinafter, each of the three combined primer pairs according to example 1 is referred as AlbioGP1, AlbioGP2 and AlbioGP3 for convenience of descriptions. Table 1 shows virtual amplification results of nucleotide sequences, SEQ ID No, amplification HPV types, sizes of amplification products, and amplification locations according to the certified primer pairs.

TABLE 1
Probes or General Primes for amplification of HPV DNA

| | | | |
|---|---|---|---|
| SEQ | | | |
| ID | AMPLIFICATION | SIZE | POSTION |

| | | | | | |
|---|---|---|---|---|---|
| PRIMER | SEQUENCE | No. | HPV TYPES | (bp) | (HPV 16) |

| | | | | | |
|---|---|---|---|---|---|
| AlbioGP1 | 5′-GATGGTGATATGGTAGAT | 1* | 1a, 2a, 3, 4, 6b, 7, 10, 11, 13, 16, 16r, | 252˜ | 6226˜6548 |
| | ACAGGATTTGG-3′ | | 18, 26, 27, 28, 30, 31, 32, 33, 34, 35 | 347 | |
| | 5′-GCGTCAGAGGTTACCATAG | 8** | 35h, 39, 40, 42, 43, 44, 45, 51, 52, | | |
| | AGCCACTAGG-3′ | | 53, 54, 55, 56, 57, 58, 59, 60, 61, 65, | | |
| | | | 66, 67, 68, 70, 72, 73, 74 and 77 | | |
| AlbioGP2 | 5′-GGCGATATGGTTGATACA | 2* | 1a, 2a, 3, 4, 6b, 7, 10, 11, 13, 16, 16r, | 306˜ | 6229˜6543 |
| | GGCTTTG-3′ | | 18, 26, 27, 28, 30, 31, 32, 33, 34, 35, | 339 | |
| | 5′-AGAAGTAACCATAGAGCC | 9** | 35h, 39, 40, 42, 43, 44, 45, 48, 50, 51, | | |
| | ACTAGG-3′ | | 52, 53, 54, 55, 56, 57, 58, 59, 60, 61, | | |
| | | | 65, 66, 67, 68, 70, 72, 73, and 77 | | |
| AlbioGP3 | 5′-GCACAACTATTTAATAAG | 3* | 2a, 3, 6b, 7, 10, 11, 13, 16, 16r, 18, 26, | 210˜ | 6547˜6762 |
| | CCATATTGG-3′ | | 27, 28, 29, 30, 31, 32, 33, 34, 35, 35h, | 242 | |
| | 5′-AATAAACTGTAAATCATA | 10** | 39, 40, 42, 43, 44, 45, 48, 49, 50, 51, | | |
| | TTCCTC-3′ | | 52, 53, 54, 55, 56, 58, 59, 60, 61, 62, | | |
| | | | 63, 66, 67, 70, 72, 73, 74, 75, 76 and | | |
| | | | 77 | | |

*5′ primer (forward primer);
**3′ primer (reverse primer)

Also, Table 2 shows product sizes of virtual amplification by using AlbioGP1, AlbioGP2 and AlbioGP3 to representative cervical-carcinoma related HPV types.

TABLE 2
Expected AMPLIFICATION PRODUCT SIZE OF GENERAL PRIMERS

| PRIMER | EXPECTED SIZE OF AMPLIFICATION PRODUCT (bp) | | | | | | | |
|---|---|---|---|---|---|---|---|---|
| PAIRS | HPV 16 | HPV 18 | HPV 31 | HPV 43 | HPV 2a | HPV 6b | HPV 11 | HPV 1a |
| AlbioGP1 | 323 | 323 | 323 | 323 | 317 | 323 | 323 | 330 |
| AlbioGP2 | 315 | 315 | 314 | 315 | 312 | 315 | 314 | 324 |
| AlbioGP3 | 216 | 218 | 215 | 218 | 210 | 213 | 213 | —* |

*No amplification

(3) Synthesis of Selected General PCR Primers

The above-selected three PCR general primer pairs AlbioGP1, AlbioGP2 and AlbioGP3 were synthesized. The primer pairs were synthesized by using Expedite™ 8909 Synthesizer (ABI inc.), which uses a solid-state synthesizing method based upon oligonucleotide phosphoramidite synthesizing chemical. To begin, the synthesis reactions were carried out on CPG columns that fixed a nucleoside located at 3′ end of oligonucleotide. The nucleosides were polymerized to oligonucleotide of the selected PCR primers by repeating basically detritylation reaction, coupling reaction, capping reaction and oxidation reaction. After terminating synthesis, 30% ammonia solution was added into CGP column for to remove the oligomers, and then the oligomers were dry-concentrated at 55 degree Celsius over 12 hours using Speed Vacuum to deprotect them. The concentrated oligomers were purified using reversed phase liquid chromatography and anion exchange chromatography. The purified final oligomers were quantitatively analyzed by measuring absorvancies at 260 nm of wavelength.

### Example 2

### PCR Primer Design and Synthesis

Example 2 was carried out on the same conditions and procedure in Example 1 except that designed general PCR primers were set as a nucleotide sequence of about 24±3 bps and about 33±2 bps and their amplification products of about 170˜550 bps.

In the above-conditions, finally eight general primer pairs were selected. Hereinafter, each of the eight combined primer pairs according to the present example is referred as AlbioGP3, AlbioGP4, AlbioGP5, AlbioGP6, AlbioGP7, AlbioGP8, AlbioGP9, AlbioGP10, and AlbioGP11 for convenience of descriptions. Table 3 shows virtual amplification results of nucleotide sequences, SEQ ID No, amplification HPV types, sizes of amplification products, and amplification locations according to the certified primer pairs. Also, Table 4 shows product sizes of virtual amplification by using AlbioGP5, AlbioGP6, AlbioGP7, AlbioGP8, AlbioGP9, AlbioGP10 and AlbioGP11 to representative cervical-carcinoma related HPV types.

Selected eight general primer pairs (AlbioGP4 to AlbioGP 11) were synthesized as the same conditions and procedures as example 1.

TABLE 3
Probes or General Primes for amplification of HPV DNA

| | | SEQ | | | |
|---|---|---|---|---|---|
| | | ID | | SIZE | POSITION |
| PRIMER | SEQUENCE | No. | AMPLIFICATION HPV TYPES | (bp) | (HPV 16) |
| AlbioGP4 | 5′-TTCTTCTTACGAAGGGAACAACTGTTTGT | 4*** | 3, 6b, 7, 10, 11, 13, 16, 18, 26, 29, 30, 31, 32, 33, 34, 35, 39, | 388˜ | 6379˜6773 |
| | TAGACA-3′ | | 40, 42, 43, 44, 45, 51, 52, 53, 54, 55, 56, 58, 59, 60, 61, 62, | 406 | |
| | 5′-CATAATTGAAACATAAACTGTAAATCAT | 11**** | 64, 66, 68, 70, 73, 74, 75, 76 and 77 | | |
| | ATTCCTC-3′ | | | | |
| AlbioGP5* | 5′-GATGGTGATATGGTAGATACAGGATTTGG-3′ | 1*** | 2a, 3, 6b, 7, 10, 11, 13, 16, 18, 26, 27, 28, 30, 31, 32, 33, 34, | 322˜ | 6226˜6548 |
| | 5′-GCGTCAGAGGTTACCATAGAGCCACTAGG-3′ | 8**** | 35, 39, 40, 42, 43, 44, 45, 51, 52, 53, 54, 55, 56, 57, 58, 59, | 334 | |
| | | | 60, 61, 65, 66, 67, 68, 70, 72, 73, 74 and 77 | | |
| AlbioGP6 | 5′-TGATATGGTTCATACAGGATTTGG-3′ | 5*** | 2a, 3, 6b, 10, 11, 13, 16, 18, 26, 27, 28, 29, 30, 31, 32, 33, 34, | 533˜ | 6230˜6762 |
| | 5′-AATAAACTGTAAATCATATTCCTC-3′ | 10**** | 35, 39, 40, 43, 44, 45, 48, 49, 50, 51, 52, 54, 55, 56, 57, 58, | 542 | |
| | | | 59, 61, 62, 64, 66, 67, 68, 70, 72, 73, 75, 76 and 77 | | |
| AlbioGP7** | 5′-GCACAACTATTTAATAAGCCATATTGG-3′ | 3*** | 2a, 3, 6b, 7, 10, 11, 13, 16, 18, 26, 27, 28, 29, 30, 31, 32, 33, | 210˜ | 6547˜6762 |
| | 5′-AATAAACTGTAAATCATATTCCTC-3′ | 10**** | 34, 35, 39, 40, 43, 44, 45, 51, 52, 54, 55, 56, 57, 58, 59, 61, | 242 | |
| | | | 62, 64, 66, 68, 70, 73, 74 and 77 | | |
| AlbioGP8 | 5′-TGTACCTGCTATTGGGGAACACTGGGCTAAG | 6*** | 1a, 2a, 3, 4, 6b, 7, 10, 11, 13, 16, 16r, 18, 26, 27, 28, 30, 31, | 432˜ | 6119˜6555 |
| | GG-3′ | | 32, 33, 34, 35, 35h, 39, 40, 42, 43, 44, 45, 48, 50, 51, 52, 53, | 438 | |
| | 5′-TAATTGGGAATCAGAAGTAACCATAGAGCCA | 12**** | 54, 55, 56, 57, 58, 59, 60, 61, 65, 66, 67, 68, 70, 72, 73, and | | |
| | CT-3′ | | 77 | | |
| AlbioGP9 | 5′-GAGGTGGGCCGGGGNCARCCNYT-3′ | 7*** | 2a, 3, 6b, 7, 10, 11, 13, 16, 16r, 18, 26, 27, 28, 29, 30, 31, 32, | 298˜ | 5947˜6218 |
| | 5′-AAGCCGGTGTCGACCATRTCNCCRTCYT-3′ | 13**** | 33, 34, 35, 35h, 39, 40, 42, 43, 44, 45, 48, 49, 50, 51, 52, 53, | 305 | |
| | | | 54, 55, 56, 58, 59, 60, 61, 62, 63, 66, 67, 70, 72, 73, 74, 75, | | |
| | | | 76 and 77 | | |
| AlbioGP10 | 5′-GAGGTGGGCCGGGGNCARCCNYT-3′ | 7*** | 1a, 2a, 3, 4, 6b, 7, 10, 11, 13, 16, 16r, 18, 26, 27, 28, 30, 31, | 301˜ | 5947˜6254 |
| | 5′-CCGAAGCCGGTGTCGAYCATRTCNCCRT-3′ | 14**** | 32, 33, 34, 35, 35h, 39, 40, 42, 43, 44, 45, 51, 52, 53, 54, 55, | 312 | |
| | | | 56, 57, 58, 59, 60, 61, 65, 66, 67, 68, 70, 72, 73, 74 and 77 | | |
| AlbioGP11 | 5′-TTCTTCTTACGAAGGGAACAACTGTTTGTTAG | 4*** | 1a, 2a, 3, 4, 6b, 7, 10, 11, 13, 16, 16r, 18, 26, 27, 28, 30, 31, | 171˜ | 6379˜6548 |
| | ACA-3′ | | 32, 33, 34, 35, 35h, 39, 40, 42, 43, 44, 45, 50, 51 ,52, 53, | 172 | |
| | 5′-GCGTCAGAGGTTACCATAGAGCCACTAGG-3′ | 8**** | 54, 55, 56, 57, 58, 59, 60, 61, 65, 66, 67, 68, 70, 72, 73, and | | |
| | | | 77 | | |

*Substantially the same as AlbioGP1;
**Substantially the same as AlbioGP2;
***5′ primer (forward primer);
****3′ primer (reverse primer);

TABLE 4
Expected AMPLIFICATION PRODUCT
SIZE OF GENERAL PRIMERS

| | EXPECTED SIZE OF AMPLIFICATION PRODUCT (bp) | | | | | | |
|---|---|---|---|---|---|---|---|
| PRIMER | | | | | HPV | HPV | HPV |
| PAIRS | HPV 16 | HPV 18 | HPV 31 | HPV 43 | 2a | 6b | 11 |
| AlbioGP4 | 397 | 400 | 397 | 391 | 395 | 397 | 392 |
| AlbioGP5 | 325 | 325 | 325 | 325 | 322 | 323 | 325 |
| AlbioGP6 | 534 | 537 | 534 | 537 | 539 | 531 | 531 |
| AlbioGP7 | 215 | 215 | 213 | 214 | 214 | 215 | 215 |
| AlbioGP8 | 438 | 438 | 438 | 438 | 432 | 438 | 438 |
| AlbioGP9 | 298 | 298 | 298 | 298 | 297 | 293 | 297 |
| AlbioGP10 | 301 | 301 | 301 | 301 | 302 | 301 | 301 |
| AlbioGP11 | 171 | 172 | 172 | 171 | 171 | 172 | 172 |

### Example 3

### PCR Amplification by General Primers

Synthesized 3 primer pairs (AlbioGP1, AlbioGP2, and AlbioGP3 of example 1 were used to amplify HPV 16-infected cell-line Caski (ATCC CRL-1550), HPV 18-infected cell-line HeLa (ATCC CCL-2) and HPV non-infected cell-line K-562)KCLB-10243, korean Cell Line Bank) as a target by PCR.

Each of Caski, HeLa and K-562 was cultured according to manufacture's instructions and then 0.25% trypsin solution was added into Caski and HeLa to centrifugate the cultured cell-lines from culturing flasks. Each of the cultured Caski, HeLa and K-562 was washed once with Dulbecco's phosphated buffer saline (Gibco Inc.) and measured cell numbers on microscope. Then 2×10⁴ cells from the cultured cell-lines were suspended with 230 μL(micro liter) of D.W. and were heated at 100° c. (degree Celsius) for 15 min. to extracted cellular DNAs. Extracted cellular DNAs were cooled at room temperature and centrifugated at 12,000 rpm for 5 minutes. Supernatants obtained from the centrifugated were utilized as template DNA solutions.

PCR reaction was accomplished with the DNA solutions and PCR reaction solutions (TaKaRa Co., LTD., Japan). PCR solution was consisted of 2.5 μL of 10×buffer, 3.75 μL of 10 mM MgCl₂, 0.5 μL of 10 mM dNTPs, 0.5 μL (5 units) of Taq polymerase, 1 μL (50 pmoles) of each of general primer pairs synthesized in Example 1 and 11.5 μL of each of template DNAs. After the PCR solution was pre-heated at 94° C. for 5 min., amplification was performed for 49 cycles of denaturing at 94° C. for 1 min., annealing at 42° C. for 1 min., and extension at 72° C. for 1 min., followed final heating 72° C. for 5 min. For HPV detection, each of 5 μL of the final amplification reactions with DNA size standard markers was transferred to 2% agarose gel, which was labeled with 0.00005% ethidium bromide solution, for electrophoresis. By comparing a band size showed in each lane of gel with the expected amplification size of table 1 in example 1, valid bands were certified.

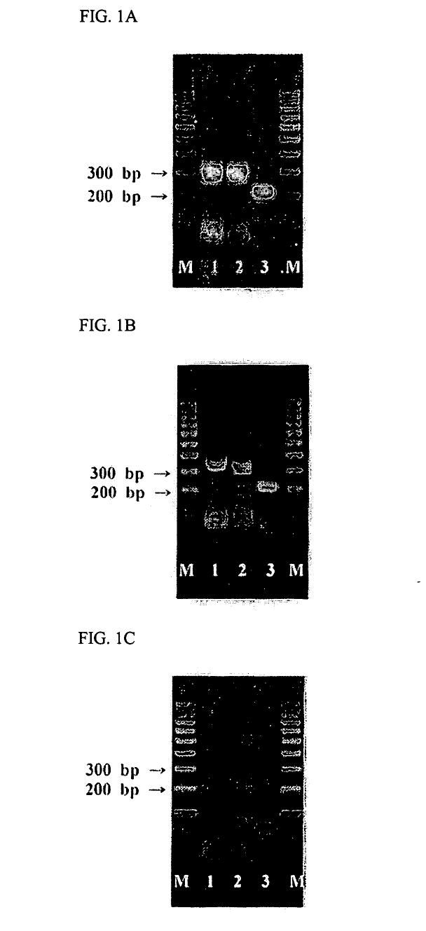

FIGS. 1A to 1C are respectively photographs of ethidium bromide-stained agarose gel electrophoresis showing PCR results when AlbioGP1, AlbioGP2 and AlbioGP3 synthesized in Example 1 were used to amplify each of HPV-16 infected cell-line Caski, HPV-18 infected cell-line HeLa and HPV non-infected cell-line K562. In the drawings, M stands for standard size marker, the numbers at the side indicate marker sizes and each of lane 1, lane 2 and lane 3 at the bottom indicates a used AlbioGP 1, AlbioGP2 and AlbioGP3.

As shown in the drawings, Each of AlbioGP1, AlbioGP2, and AlbioGP3 make an amplification products, which are identical to expected results of table 2, to HPV 16 infected cell-line Caski (FIG. 1A) and HPV 18 infected cell-line HeLa (FIG. 1B), but did not make an amplification product to HPV non-infected cell-line K-562 (FIG. 1C).

### Example 4

### PCR Amplification by General Primer Pairs

Synthesized 8 primer pairs (AlbioGP4, AlbioGP5, AlbioGP6, AlbioGP7, AlbioGP8, AlbioGP9, AlbioGP10and AlbioGP3) of Example 2 were used to amplify HPV 16-infected cell-line Caski (ATCC CRL-1550), HPV 18-infected cell-line HeLa (ATCC CCL-2) and HPV non-infected cell-line K-562 (KCLB-10243, Korean Cell Line Bank) as a target by PCR.

Each of cultured Caski, HeLa and K-562 according to manufacture's instructions was washed once with Dulbecco's phosphated buffer saline (Gibco Inc.) and measured cell numbers on microscope. Then 2×10⁶ cells from the cultured cell-lines were separated and purified using “Genomic Isolation Kit (Cat. No. K-3032, Bioneer Inc.)” and melted in 200 L(micro liter) of D.W to obtain final template DNA solutions.

PCR reaction was accomplished as the same conditions and procedures as Example 3 except that 8.0 μL of each of template DNAs was contained in total 25 μL of PCR reaction solution.

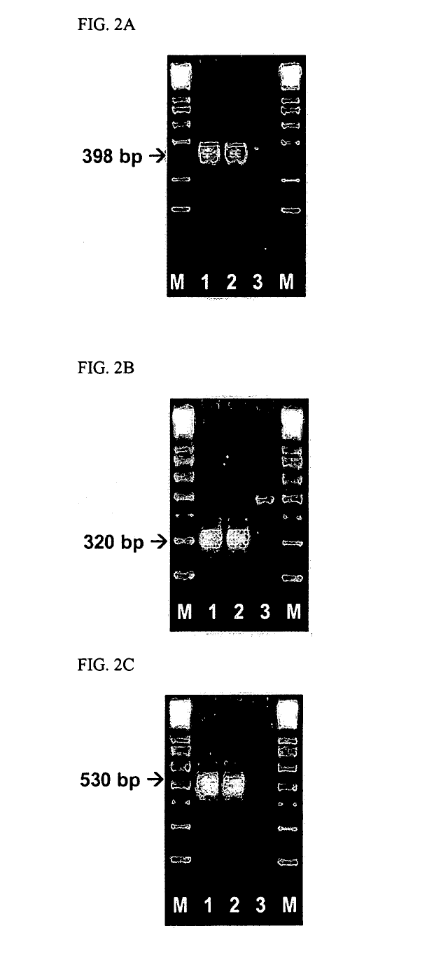

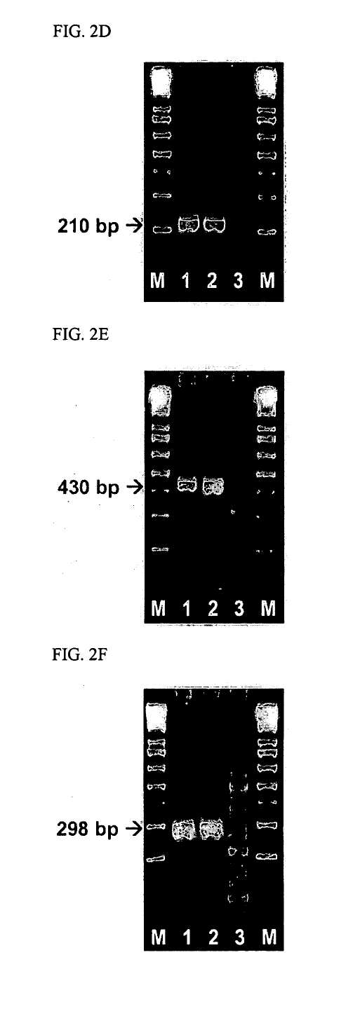

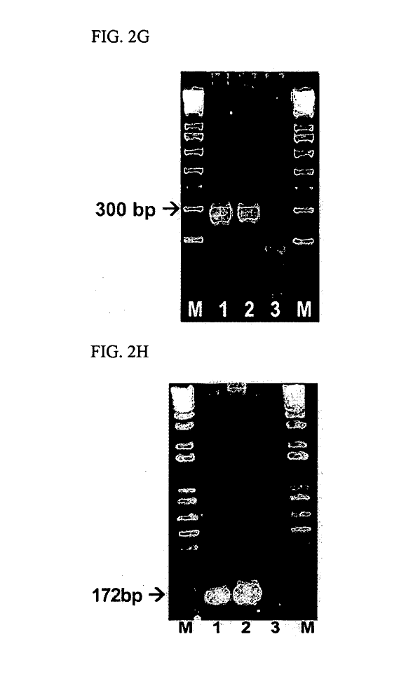

FIGS. 2A to 2H are respectively photographs of ethidium bromide-stained agarose gel electrophoresis showing PCR results when AlbioGP4, AlbioGP5, AlbioGP6, AlbioGP7, AlbioGP8, AlbioGP9, AlbioGP10 and AlbioGP 11 synthesized in Example 1 were used to amplify each of HPV-16 infected cell-line Caski, HPV-18 infected cell-line HeLa and HPV non-infected cell-line K562. In the drawings, M stands for standard size marker, the numbers at the side indicate sizes of amplification products and each of lane 1, lane 2 and lane 3 at the bottom indicates targeted cell-lines CaSki, HeLa and K562.

FIGS. 2A to 2H show bands which are as the same as expected result of table 4 to targeted HPV 16 infected cell-line Caski (lane 1) and HPV 18 infected cell-line HeLa (lane 2), i.e., AlbioGP4, 398bp, in FIG. 2A; AlbioGP5, 320bp, in FIG. 2B; AlbioGP6, 530bp, in FIG. 2C; AlbioGP7, 210bp, in FIG. 2D; AlbioGP8, 430bp, in FIG. 2E; AlbioGP9, 298bp, in FIG. 2F; AlbioGP10, 300bp, in FIG. 2G; and AlbioGP11, 172bp, in FIG. 2H. But, every general primer pair did not make an amplification product to HPV non-infected cell-line K562 (lane 3).

### Example 5

### PCR Amplification Using HPV Containing Various Plasmids

In this example, general primer pairs AlbioGP1, AlbioGP2 and AlbioGP3 of Example 1 were used to amplify E. coli strains containing plasmid DNA of various HPV types as a target. These strains were ATCC 45021 (HPV1a), ATCC (HPV 2a), ATCC 45150 (HPV 6b), ATCC 45151 (HPV 11), ATCC 45113 (HPV 16), ATCC 45152 (HPV 18), ATCC 65446 (HPV 31) and ATCC 40338 (ATCC 43).

Each E.coli strain containing plasmid inserted DNA of each HPV type was cultured with shaking on 37 LB liquid culture medium and separated and purified using “Qiafilter Plasmid Maxi Kit (Cat. No. 12263, QIAGEN Corp.)” to modulate a concentration of 10 pg/μL. These concentration-modulated strains are utilized as template DNA solution.

PCR amplification was accomplished to each of the template DNA using each general primer pair as the same reaction composition and procedures as Example 3. By comparing a band size showed in each lane of gel with the expected amplification size of table 2 in example 1, valid bands were certified.

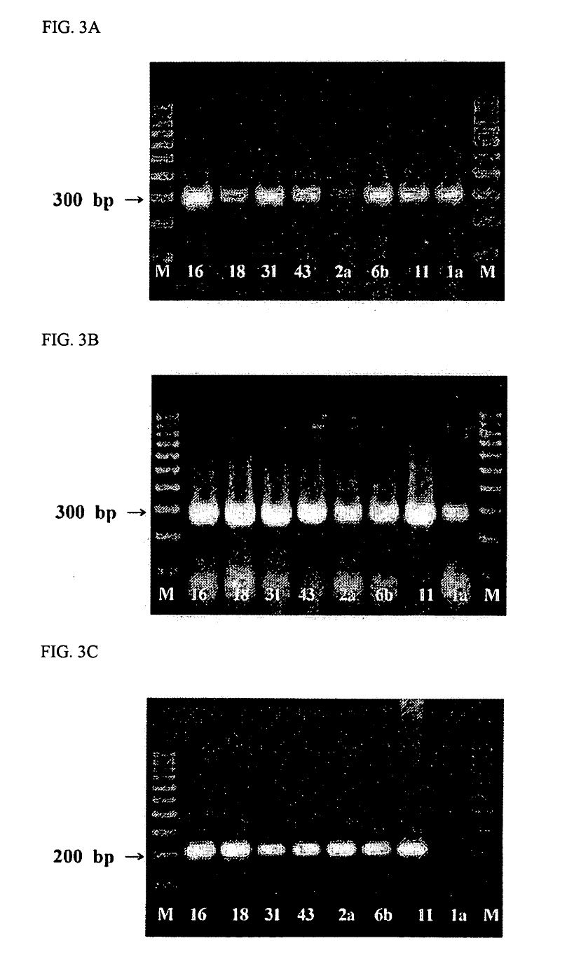

FIGS. 3A to 3C are respectively gel electrophoresis photographs showing PCR results when AlbioGP1, AlbioGP2, and AlbioGP3 of Example 1 were used to amplify E.coli strains containing plasmid DNA of specific HPV type. In the drawings, M stands for standard size marker, the numbers at the side indicate marker sizes and the numbers at the bottom indicate HPV types inserted in the strains.

FIGS. 3A to 3C show bands which are as the same as expected result of table 2 to E. coli strains containing specific HPV DNA, i.e. AlbioGP1, about 300˜320b to targeted every strain (FIG. 3A); AlbioGP2, about 300˜320bp to targeted every strain (FIG. 3B); and AlbioGP3, about 200˜220bp to targeted every strain except a strain containing plasmid DNA of HPV 1a (FIG. 3C).

### Example 6

### PCR Amplification Using HPV Containing Various Plasmids

In this example, seven general primer pairs AlbioGP4, AlbioGP5, AlbioGP6, AlbioGP7, AlbioGP8, AlbioGP9, AlbioGP10, AlbioGP10 of Example 2 were used to amplify E.coli strains containing plasmid DNA of various HPV types as a target. Targeted strains were same as Example 5 except that ATCC 45021 containing HPV 1a DNA was not used.

Each E.coli strain containing plasmid inserted DNA of each HPV type was cultured with shaking on 37 LB liquid culture medium and separated and purified using “Qiafilter Plasmid Maxi Kit (Cat. No. 12263, QIAGEN Corp.)” to modulate a concentration of 100 ng/μL. These concentration-modulated strains are utilized as template DNA solution.

PCR amplification was accomplished to each of the template DNA using each general primer pair as the same reaction composition and procedures as Example 4. By comparing a band size showed in each lane of gel with the expected amplification size of table 4 in example 2, valid bands were certified.

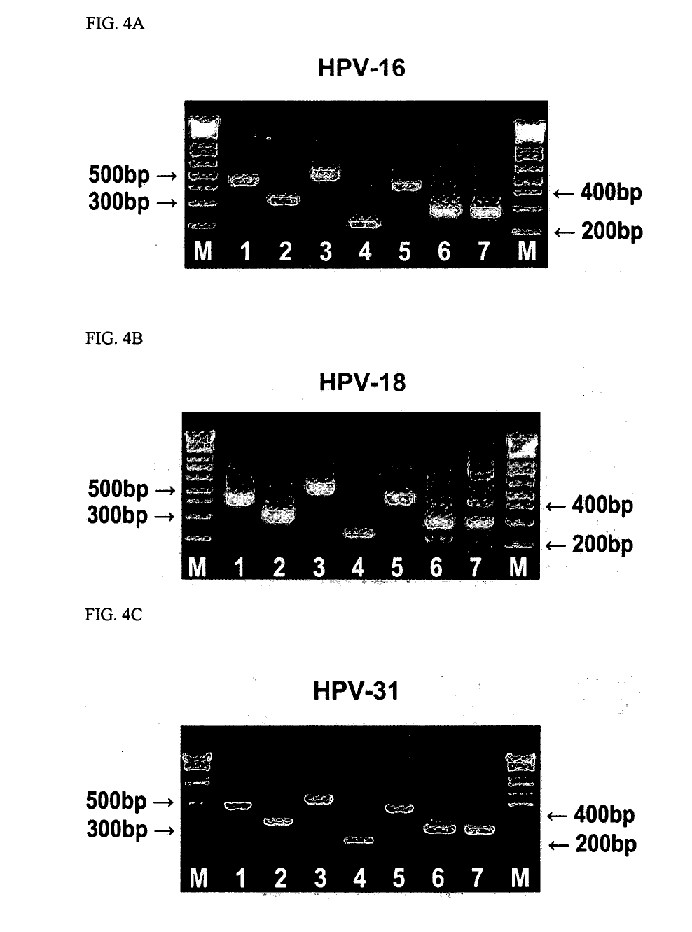

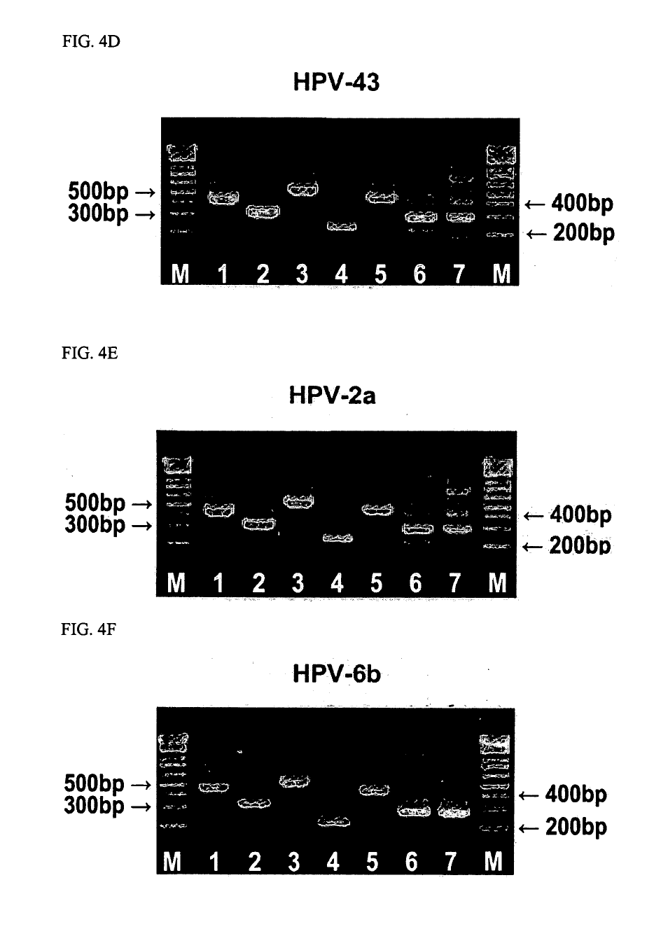

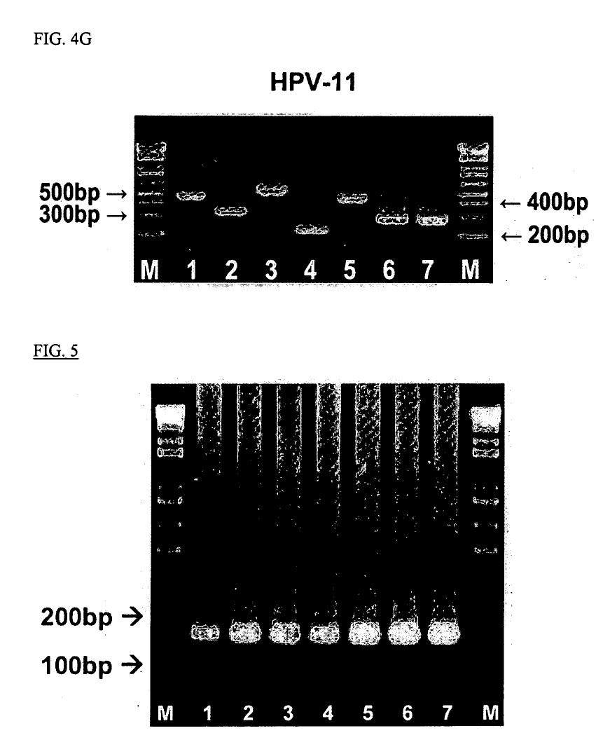

FIGS. 4A to 4G are respectively gel electrophoresis photographs showing PCR results when AlbioGP4, AlbioGP5, AlbioGP6, AlbioGP7, AlbioGP8, AlbioGP9, and AlbioGP10 of Example 2 were used to amplify each of E.coli strain containing HPV DNA of type 16 (ATCC 45113 strain), type 18 (ATCC 45142 strain), type 31 (ATCC 65446 strain), type 43 (ATCC 40338 strain), type 2a (ATCC 45202 strain), type 6b (ATCC 45140 strain) and type 11 (ATCC 45152 strain). In the drawings, M stands for standard size marker, the numbers at sides indicate marker sizes and lane 1, lane 2, lane 3, lane 4, lane 5, lane 6, and lane 7 at the bottom indicate respectively used general primer pair AlbioGP4, AlbioGP5, AlbioGP6, AlbioGP7, AlbioGP8, AlbioGP9 and AlbioGP10.

FIGS. 4A to 4G show bands which are as the same as expected result of table 4 to E. coli strains containing a specific HPV DNA, i.e. AlbioGP4, about 380˜410bp to targeted every strain, in lane 1 each of FIGS. 4A to 4G; AlbioGP5, about 310˜330bp to targeted every strain, in lane 2 each of FIGS. 4A to 4G; AlbioGP6, about 520˜550bp to targeted every strain, in lane 3 each of FIGS. 4A to 4G; AlbioGP7, about 210˜230bp to targeted every strain, in lane 4 each of FIGS. 4A to 4G; AlbioGP8, about 430˜450bp to targeted every strain, in lane 5 each of FIGS. 4A to 4G; AlbioGP9, about 280˜310bp to targeted every strain, in lane 6 each of FIGS. 4A to 4G; and AlbioGP10, about 290˜310bp to targeted every strain, in lane 7 each of FIGS. 4A to 4G.

### Example 7

### PCR Amplification Using HPV Containing Various Plasmids

In this example, general primer pair AlbioGP11 of Example 2 were used to amplify E.coli strains containing plasmid DNA of various HPV types as a target. Targeted strains were same as Example 6.

Each E.coli strain containing plasmid DNA of each HPV type was extracted to template DNA solution and PCR amplification was accomplished as the same conditions and procedures as Example 6.

FIGS. 5 is a gel electrophoresis photograph showing PCR results when AlbioGP11 was used to amplify each of E.coli strain containing a specific HPV type. In the drawings, M stands for standard size marker, the numbers at the side indicate marker sizes and lane 1, lane 2, lane 3, lane 4, lane 5, lane 6, and lane 7 at the bottom indicate each of targeted E. coli strain containing HPV DNA of type 16 (ATCC 45113 strain), type 18 (ATCC 45142 strain), type 31 (ATCC 65446), type 43 (ATCC 40338 strain), type 2a (ATCC 45202 strain), type 6b (ATCC 45140 strain) and type 11 (ATCC 45151).

FIG. 5 shows bands that are identical as expected results of table 4 to every E. coli strain containing a specific HPV DNA. That is, AlbioGP11 made amplification products of about 160˜180bp to each of ATCC 45113 strain containing HPV 16 DNA, ATCC 45142 strain containing HPV 18 DNA, ATCC 65446 strain containing HPV 31 DNA, ATCC 40338 strain containing HPV 43 DNA, ATCC 45202 strain containing HPV 2a DNA, ATCC 45140 strain containing HPV 6b, and ATCC 45151 strain containing HPV11 DNA.

### Comparative Example 1

### Cytological Examination for HPV Infection

In this comparative example, clinical samples obtained from subjects were examined according to conventional cytological tests.

Under control of charging clinicians, cytological tests such as colopscopy test, cervicography test, biopsy test and Pap smear were accomplished into clinical samples obtained from randomly selected 30 subjects who had visited to gynecological caner center in affiliation with “College of Medicine Pochon CHA University” hospital. As shown in Table 4 which indicates results of cytological tests, subjects 1, 2, 3, 4, 5, 6, 7, 9, 10, 12, 13, 14, 18, 19, and 20 were diagnosed as SCC (squamous cell carcinoma), subject 6 was diagnosed as “CIS (carcinoma in situ) which is a pre-stage of cervical cancer, and subjects 8, 11, 15, 16 and 17 were diagnosed “SIL (squamous intraepithelial lesion) which is also a pre-stage of cervical carcinoma. Those 20 subjects are classified as abnormal patients.

Other subjects are included in cervical-cancer non-related patients since subjects 21, 22, 23, 26 and 27 have normal cervical cells, subjects 24, 28, 29 and 30 have only myoma which is not related to cervical cancer and subject 25 have only adenomyosis which is not related to cervical cancer.

TABLE 5
Clinical Result according to Cytological Tests

| Subject No. | Test Result |
|---|---|
| 1 | SCC* |
| 2 | SCC |
| 3 | SCC |
| 4 | SCC |
| 5 | SCC |
| 6 | CIS** |
| 7 | SCC (recur.) |
| 8 | SIL*** |
| 9 | SCC |
| 10 | SCC |
| 11 | SIL |
| 12 | SCC |
| 13 | SCC |
| 14 | SCC (recur.) |
| 15 | SIL |
| 16 | SIL |
| 17 | SIL |
| 18 | SCC |
| 19 | SCC |
| 20 | SCC |
| 21 | Normal |
| 22 | Normal |
| 23 | Normal |
| 24 | UM**** |
| 25 | UM |
| 26 | Normal |
| 27 | Normal |
| 28 | UM |
| 29 | UM |
| 30 | UM |

*Squamous cell carcinoma;
**Carcinoma in situ;
***Squamous intraepithelial lesion
****Uretus myoma

### Comparative Example 2

### Hybrid Capture Test and DNA Chip Test

In this comparative example, some samples obtained from subjects of comparative example 1 were tested for diagnosing HPV infection or not based upon hybrid capture test II and DNA chip test. Table 6 shows test results according to hybrid capture II and DNA chip.

Sample obtained from subjects 1, 6, 8, 9, 21, 22, 23, 24, 25 and 28 were tested using hybrid capture II (DIGENE Corp.). Subjects 1, 6, 8 and 9 were diagnosed as cervical cancer positive and subjects 21, 22, 23, 24, 25 and 28 were cervical cancer negative according to the hybrid capture II.

Samples obtained from subjects 2, 3, 4, 5, 10, 11, 12, 18, 19 and 20 were tested using DNA chip (BIOMEDRAB Co., LTD, Korea). Each sample from subjects 2, 3, 4, 5, 10, 11, 12, 18, 19 and 20 was diagnosed as infected by HPV types 16, 16, 16, 16, 16, 58, 16, 16, 58and 58. Those results of hybrid capture test and DNA chip test are identical to the result of comparative example 1.

TABLE 6
Clinical Result using hybrid capture and DNA chip

HPV DNA Diagnose Test Result

| Subject No. | Hybrid Capture II (High/Low) | DNA chip |
|---|---|---|
| 1 | Positive/Negative | —* |
| 2 | — | 16 |
| 3 | — | 16 |
| 4 | — | 16 |
| 5 | — | 16 |
| 6 | Positive/Negative | — |
| 8 | Positive/Negative | — |
| 9 | Positive/Negative | — |
| 10 | — | 16 |
| 11 | — | 58 |
| 12 | — | 16 |
| 18 | — | 16 |
| 19 | — | 58 |
| 20 | — | 58 |
| 21 | Negative/Negative | — |
| 22 | Negative/Negative | — |
| 23 | Negative/Negative | — |
| 24 | Negative/Negative | — |
| 25 | Negative/Negative | — |
| 28 | Negative/Negative | — |

*No test

### Example 8

### Diagnosis of Cervical Carcinoma by General Primer Pairs

In this example, samples obtained from abnormal subjects of comparative example 1 as a target DNA were amplified using general primer pairs AlbioGP 1, AlbioGP2 and AlbioGP3 of example 1 and general primer set MY09 (5′-CGTCCMARRGGAWACTGATC-3′, SEQ ID No: 15)/MY11 (5′-GCMCAGGGWCATAAYAATGG-3′, SEQ ID No: 16) conventionally used for HPV detection in PCR followed electrophoresis. DNA Samples were obtained from abnormal subjects as conventional procedures and PCR and electrophoresis were carried out as identical conditions and procedures as example 3.

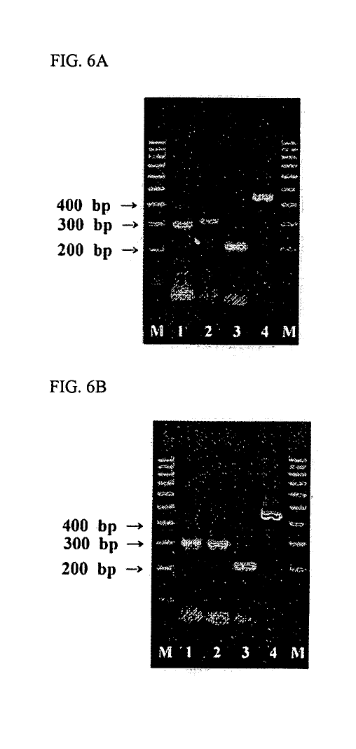

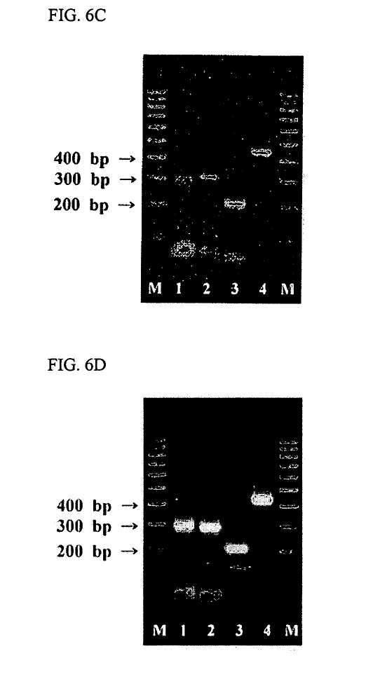

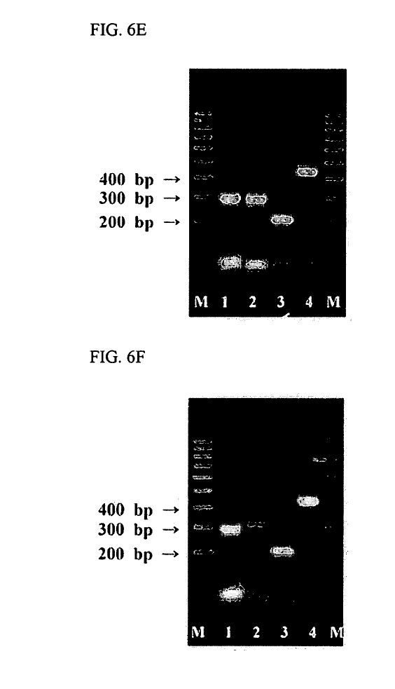

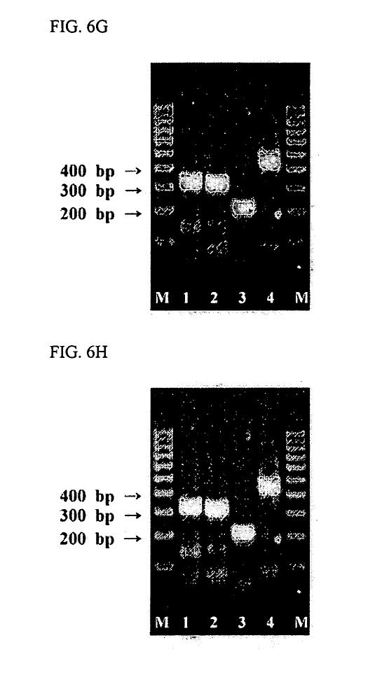

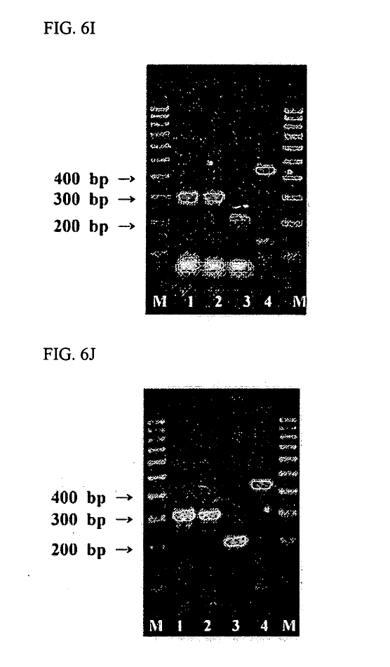

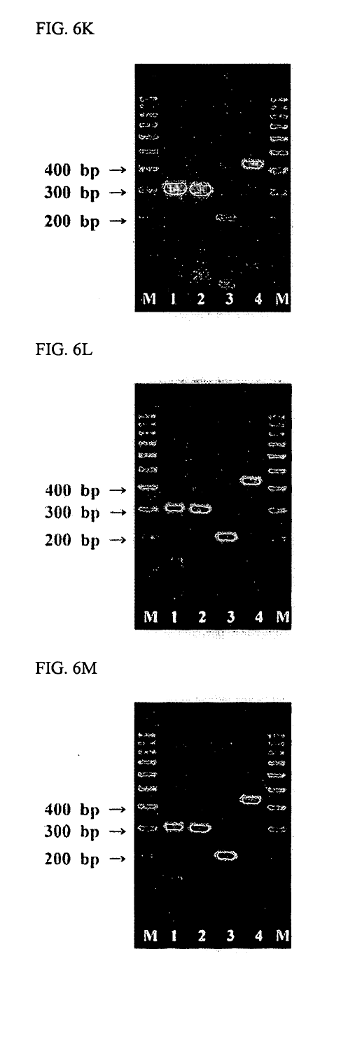

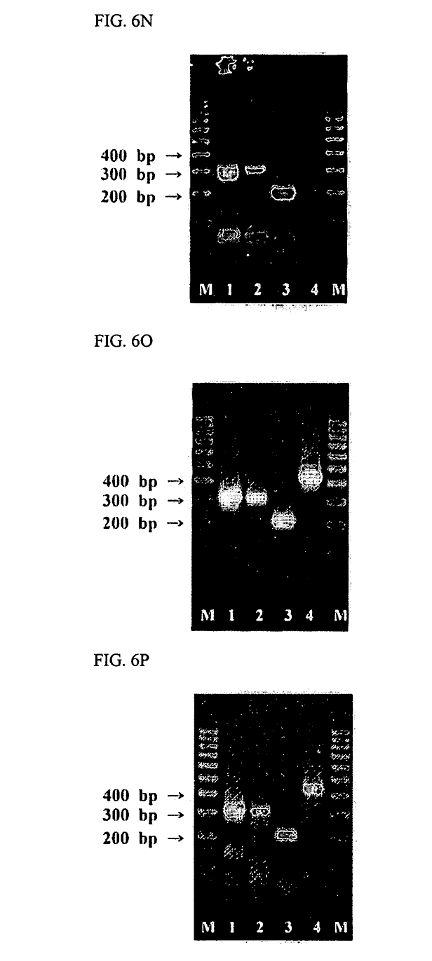

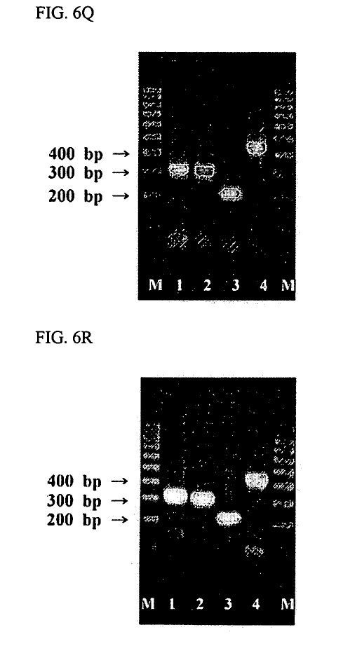

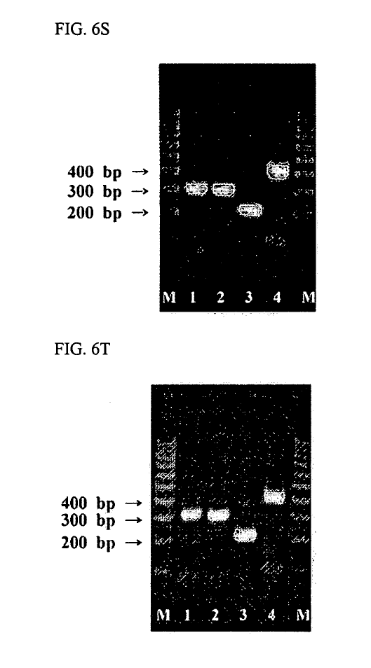

FIGS. 6A to 6T are respectively gel electrophoresis photographs showing PCR results when 3 primer pairs synthesized according to example 1 and conventionally used primer pair for amplifying HPV DNA were used to amplify each of DNA samples obtained from cervical-cancer related, abnormal subjects (subjects 1 to 20) of comparative example 1.

In the drawings, M stands for a standard size marker, the numbers at the side indicate marker sizes and each of lane 1, lane 2, lane 3 and lane 4 at the bottom indicates a used primer pair AlbioGP1, AlbioGP2, AlbioGP3 and MY09/MY11 set.

FIGS. 6A to 6G show bands which are as the same as expected result of table 4 to each of DNA samples obtained from cervical-related subjects, i.e. AlbioGP1, about 310˜320bp, in lane 1 each of FIGS. 6A to 6T; AlbioGP2, about 300˜320bp, in lane 2 each of FIGS. 6A to 6T; and AlbioGP3, about 210˜230bp, in lane 3 each of FIGS. 6A to 6T. Especially, DNA sample obtained from cervical cancer recurrent patient (subject 14 of comparative example 1) as a target was amplified in case of using AlbioGP1, AlbioGP2 and AlbioGP3 primer pairs, not MY09/MY11 primer set as shown in FIG. 6N.

### Example 9

### Diagnosis of Cervical Carcinoma by General Primer Pairs

In this example, samples obtained from normal subjects of comparative example 1 as a target DNA were amplified as identical conditions and procedure as example 8.

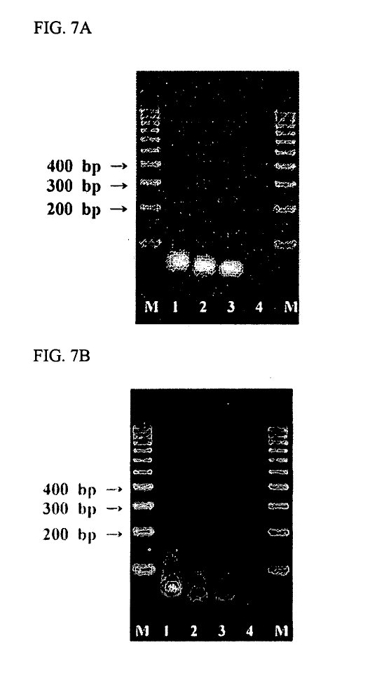

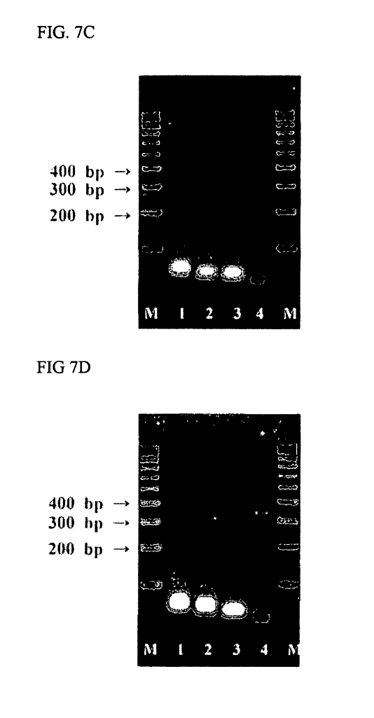

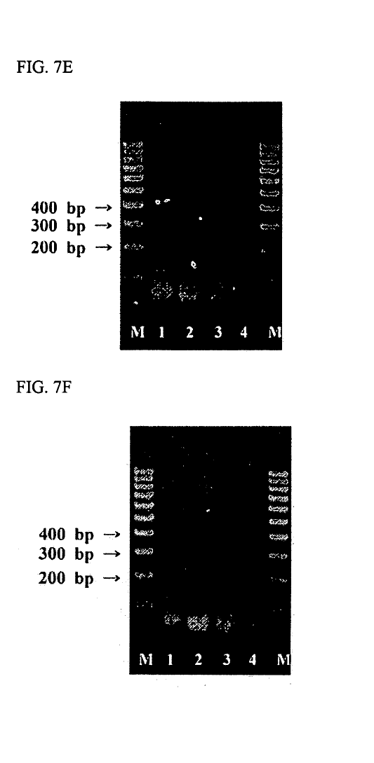

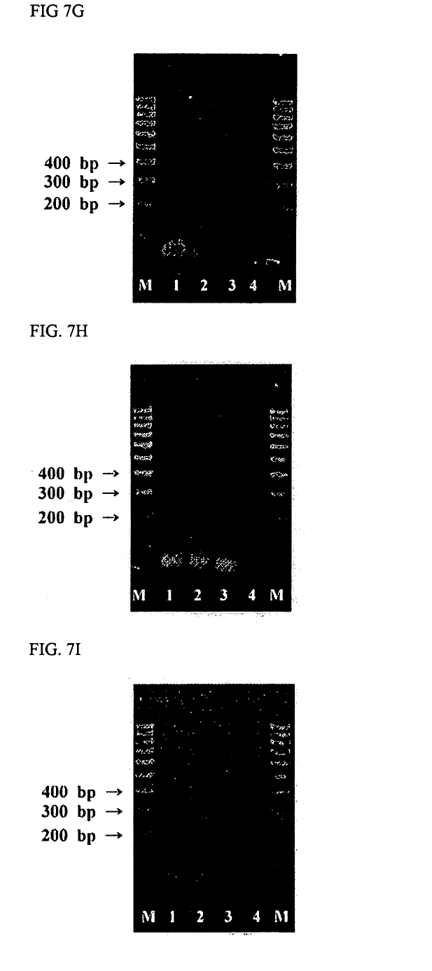

FIGS. 7A to 7J are respectively gel electrophoresis photographs showing PCR results when AlbioGP1, AlbioGP2, AlbioGP3, and conventionally used primer pair MY09/NY11 primer set were used to amplify each of DNA samples obtained from cervical cancer non-related, normal subjects (subjects 21 to 30) of comparative example 1.

In the drawings, M stands for a standard size marker, the numbers at the side indicate marker sizes and each of lane 1, lane 2, lane 3 and lane 4 at the bottom indicates a used primer pair AlbioGP1, AlbioGP2, AlbioGP3 and MY09/MY11 set.

As shown in FIGS. 6A to 6G, DNA samples obtained from normal subjects were not amplified in case of using each of AlbioGP1, AlbioGP2 and AlbioGP3 of the present invention and MY09/MY11 primer set.

### Comparative Example 3

### Cytological Examination for HPV Infection

In this comparative example, clinical samples obtained from other 30 subjects were examined according to conventional cytological tests as identical procedure as comparative example 1.

Table 7 shows results of cytological tests into 30 subjects. As shown in Table 7, subjects 1, 2, 5, 7, 8, 13, 14, 16, 17, and 18 were diagnosed as HSIL (high squamous intraepithelial lesion), subjects 6, 9, 11, 12, 19, and 20 was diagnosed as “ASCUS (Atypical squamous cell undetermined significance), both of which are regarded as a pre-stage of cervical carcinoma, and subjects 3, 4, 10 and 15 were diagnosed “SCC (squamous cell carcinoma). Those 20 subjects are classified as abnormal patients. On the other hand, other subjects are included in cervical-cancer non-related normal patients.

TABLE 5
Clinical Result according to Cytological Tests

| Subject No. | Test Result |
|---|---|
| 1 | HSIL* |
| 2 | HSIL |
| 3 | SCC** |
| 4 | SCC |
| 5 | HSIL |
| 6 | ASCUS*** |
| 7 | HSIL |
| 8 | HSIL |
| 9 | ASCUS |
| 10 | SCC |
| 11 | ASCUS |
| 12 | ASCUS |
| 13 | HSIL |
| 14 | HSIL |
| 15 | SCC |
| 16 | HSIL |
| 17 | HSIL |
| 18 | HSIL |
| 19 | ASCUS |
| 20 | ASCUS |
| 21 | Normal |
| 22 | Normal |
| 23 | Normal |
| 24 | Normal |
| 25 | Normal |
| 26 | Normal |
| 27 | Normal |
| 28 | Normal |
| 29 | Normal |
| 30 | Normal |

*High squamous intraepithelial carcinoma;
**Squamous cell lesion; and
***Atypical squamous cell undetermined significance

### Comparative Example 4

### Hybrid Capture Test and DNA Chip Test

In this comparative example, DNA samples obtained from subjects of example 1 were tested for diagnosing HPV infection or not based upon hybrid capture.

Samples obtained from subjects 1 to 30 were tested using hybrid capture II method (DIGENE Corp.). Each sample from cervical-carcinoma related, abnormal subjects 1˜11 and 11˜19 was diagnosed as cervical cancer positive and each sample of cervical-carcinoma non-related, normal subjects was cervical carcinoma negative. These hybrid capture II based test results are identical to the result of comparative example 1. However, sample from abnormal subjects 12 and 20 according to comparative example 3 were diagnoised as cervical carcinoma negative.

TABLE 8
Clinical Result using hybrid capture II

| Subject | Result | Subject | | | Test |
|---|---|---|---|---|---|
| No. | (High/Low) | No. | Test Result | Subject No. | Result |
| 1 | +/− | 11 | +/− | 21 | −/− |
| 2 | +/− | 12 | −/− | 22 | −/− |
| 3 | +/− | 13 | +/− | 23 | −/− |
| 4 | +/− | 14 | +/− | 24 | −/− |
| 5 | +/− | 15 | +/− | 25 | −/− |
| 6 | +/− | 16 | +/− | 26 | −/− |
| 7 | +/− | 17 | +/− | 27 | −/− |
| 8 | +/− | 18 | +/− | 28 | −/− |
| 9 | +/− | 19 | +/− | 29 | −/− |
| 10 | +/− | 20 | −/− | 30 | −/− |

### Example 10

### Diagnosis of Cervical Neoplasia by General Primer Pairs

In this example, DNA samples obtained from abnormal subjects of comparative example 3 were amplified using general primer pairs AlbioGP4, AlbioGP5, AlbioGP6, AlbioGP7, AlbioGP9 and AlbioGP10 of example 2 in PCR followed electrophoresis for diagnosing both HPV infection and cervical neoplasia at early stage. DNA Samples were obtained from abnormal subjects as conventional procedures and PCR and electrophoresis were carried out as identical conditions and procedures as example 4.

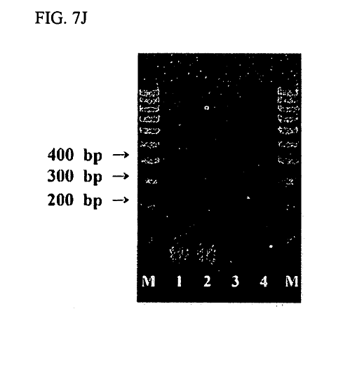

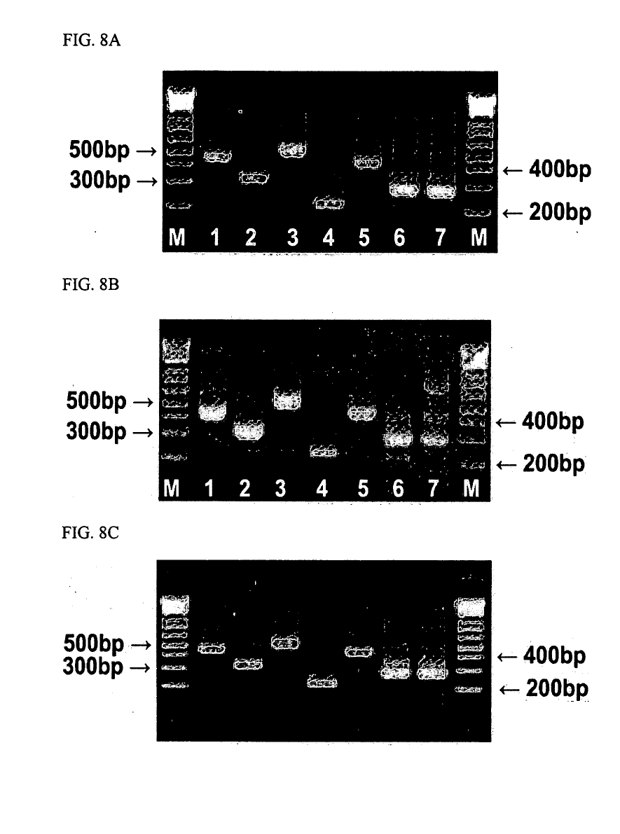

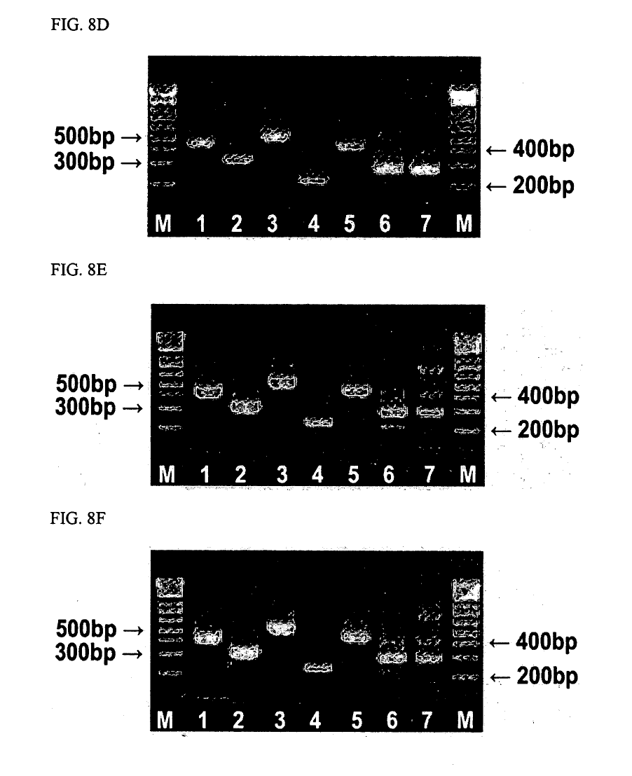

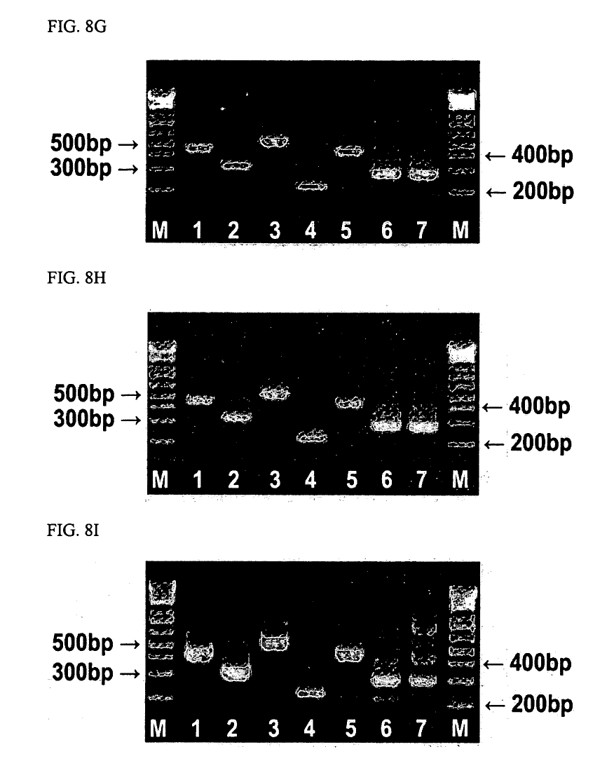

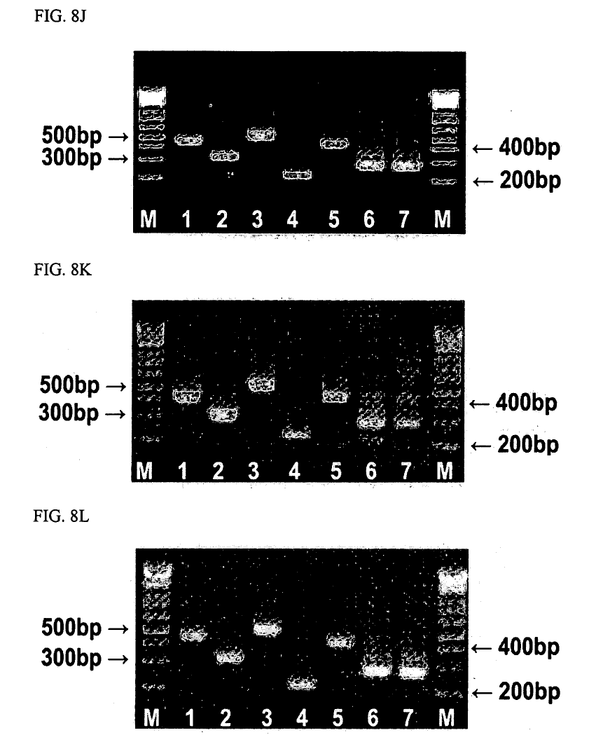

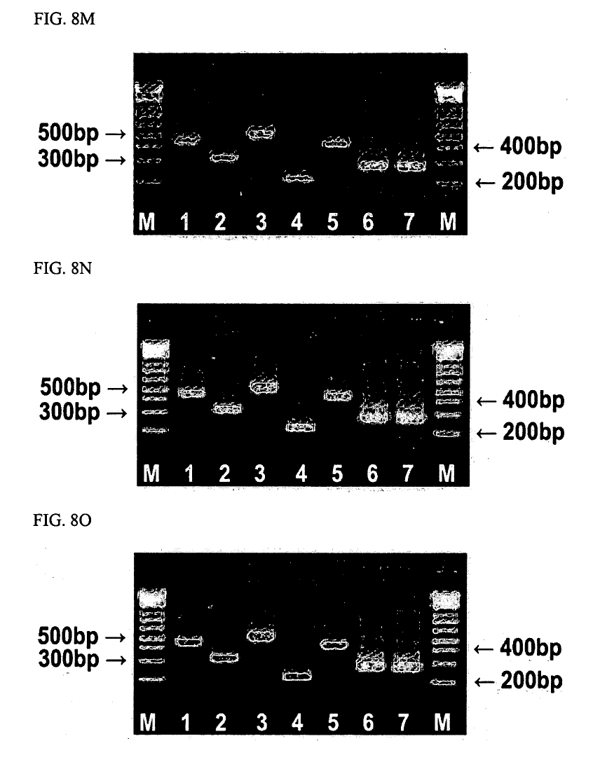

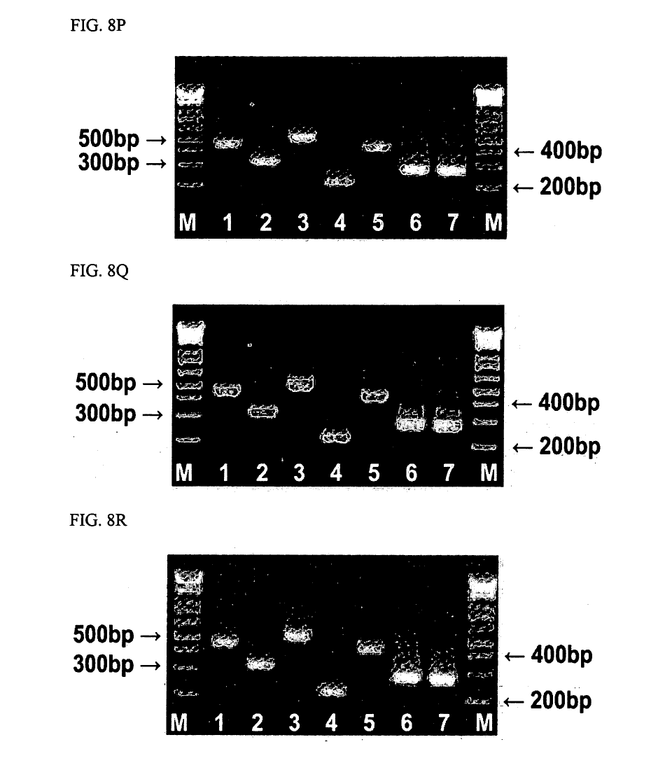

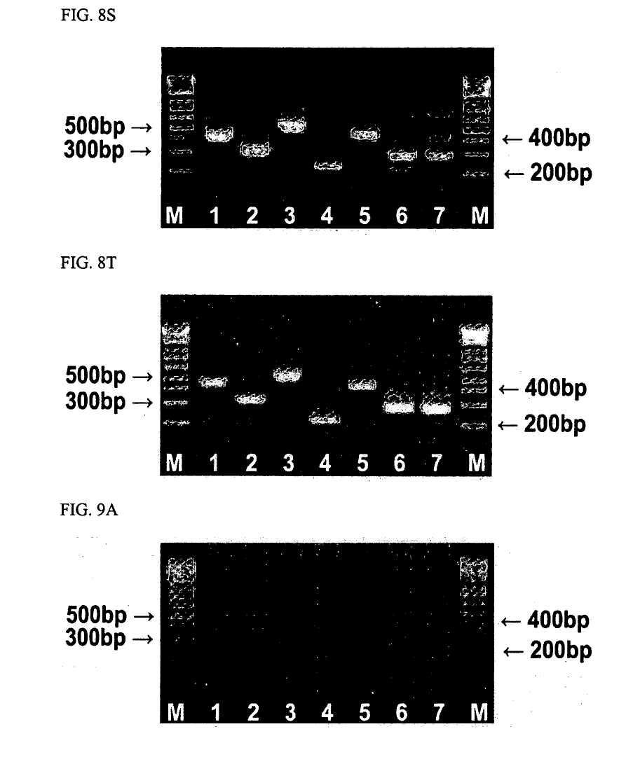

FIGS. 8A to 8T are respectively gel electrophoresis photographs showing PCR results when 7 primer pairs synthesized according to example 2 were used to amplify each of DNA samples obtained from cervical-cancer related, abnormal subjects (subjects 1 to 20) of comparative example 3.

In the drawings, M stands for a standard size marker, the numbers at the side indicate marker sizes and each of lane 1, lane 2, lane 3, lane 4, lane 5, lane 6 and lane 7 at the bottom indicates a used primer pair AlbioGP4, AlbioGP5, AlbioGP6, AlbioGP7, AlbioGP8, AlbioGP9 and AlbioGP10.

FIGS. 8A to 8T show bands which are as the same as expected result of table 4 to each of DNA samples obtained from cervical-neoplasia related subjects, i.e. AlbioGP4, about 310˜320bp, in lane 1 each of FIGS. 8A to 8T; AlbioGP5, about 300˜320bp, in lane 2 each of FIGS. 8A to 8T; AbioGP6, about 210˜230bp, in lane 3 each of FIGS. 8A to 8T; AlbioGP7, about ˜430bp, in lane 4 each of FIGS. 8A to 8T; AlbioGP8, about ˜444bp, in lane 5 each of FIGS. 8A to 8T; AlbioGP9, about ˜450bp, in lane 6 each of FIGS. 8A to 8T; and AlbioGP10, about ˜33bp, in lane 7 each of FIGS. 8A to 8T.

### Example 11

### Diagnosis of Normal Clinical Samples by General Primer Pairs

In this example, samples obtained from normal subjects of comparative example 3 as a target DNA were amplified as identical conditions and procedure as example 8.

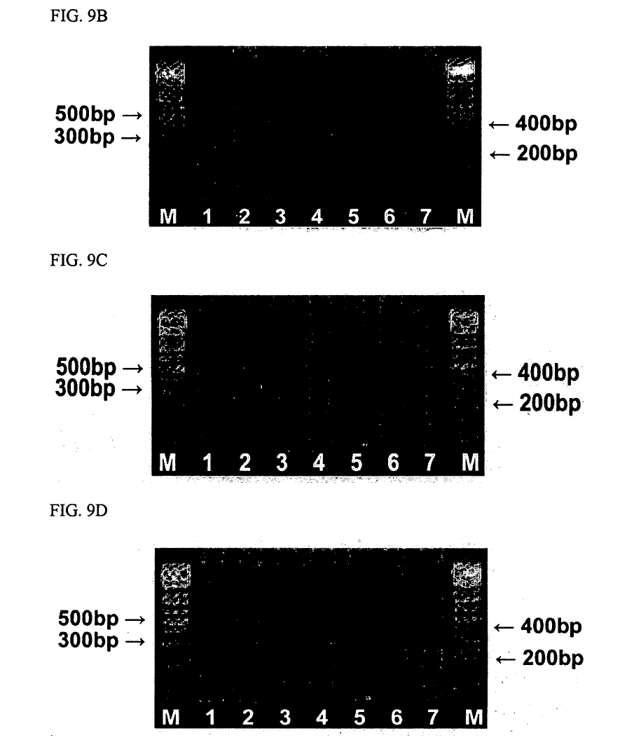

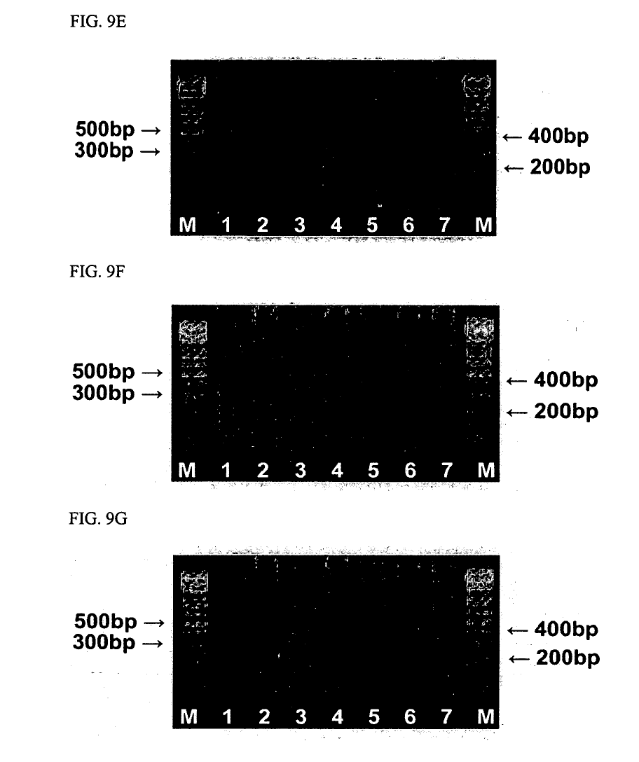

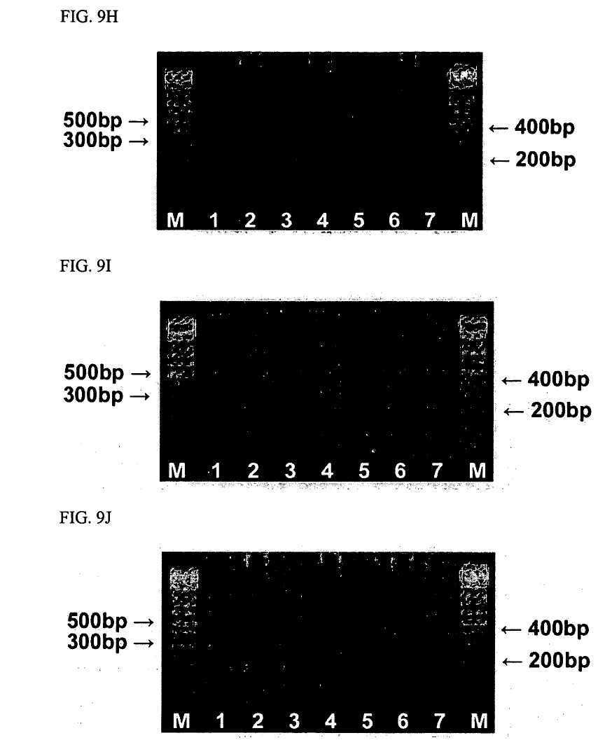

FIGS. 9A to 9J are respectively gel electrophoresis photographs showing PCR results when AlbioGP4, AlbioGP5, AlbioGP6, AlbioGP7, AlbioGP8, AlbioGP9 and AlbioGP10 were used to amplify each of DNA samples obtained from cervical cancer non-related, normal subjects (subjects 21 to 30) of comparative example 3.

In the drawings, M stands for a standard size marker, the numbers at the side indicate marker sizes and each of lane 1, lane 2, lane 3, lane 4, lane 5, lane 6 and lane 7 at the bottom indicates a used primer pair AlbioGP4, AlbioGP5, AlbioGP6, AlbioGP7, AlbioGP8, AlbioGP9 and AlbioGP10.

As shown in FIGS. 9A to 9G, DNA samples obtained from normal subjects were not amplified in case of using any of AlbioGP4, AlbioGP5, AlbioGP6, AlbioGP7, AlbioGP8, AlbioGP9 and AlbioGP10.

It will be apparent to those skilled in the art that various modifications and variations can be made in the fabrication and application of the present invention without departing from the spirit or scope of the invention. Thus, it is intended that the present invention cover the modifications and variations of this invention provided they come within the scope of the appended claims and their equivalents.

What is claimed is:

1. A general primer for amplifying and detecting Human Papillomavirus genotypes, which is an oligonucleotide selected from the group consisting of:
(a) SEQ ID NO: 1 or a sequence which is fully complementary to SEQ ID NO: 1;
(b) SEQ ID NO: 2 or a sequence which is fully complementary to SEQ ID NO: 2;
(c) SEQ ID NO: 3 or a sequence which is fully complementary to SEQ ID NO: 3;
(d) SEQ ID NO: 4 or a sequence which is fully complementary to SEQ ID NO: 4;
(e) SEQ ID NO: 5 or a sequence which is fully complementary to SEQ ID NO: 5;
(f) SEQ ID NO: 6 or a sequence which is fully complementary to SEQ ID NO: 6; and
(g) SEQ ID NO: 7 or a sequence which is fully complementary to SEQ ID NO: 7.

2. A general primer for amplifying and detecting Human Papillomavirus genotypes, which is an oligonucleotide selected from the group consisting of:
(a) SEQ ID NO: 8 or a sequence which is fully complementary to SEQ ID NO: 8;
(b) SEQ ID NO: 9 or a sequence which is fully complementary to SEQ ID NO: 9;
(c) SEQ ID NO: 10 or a sequence which is fully complementary to SEQ ID NO: 10
(d) SEQ ID NO: 11 or a sequence which is fully complementary to SEQ ID NO: 11
(e) SEQ ID NO: 12 or a sequence which is fully complementary to SEQ ID NO: 12;
(f) SEQ ID NO: 13 or a sequence which is fully complementary to SEQ ID NO: 13; and
(g) SEQ ID NO: 14 or a sequence which is fully complementary to SEQ ID NO: 14.

3. A general primer pair for use in a nucleic acid amplification process for amplifying of Human Papillomavirus genotypes, wherein a first primer is an oligonucleotide selected from the group consisting of:
(a) SEQ ID NO: 1 or a sequence which is fully complementary to SEQ ID NO: 1;
(b) SEQ ID NO: 2 or a sequence which is fully complementary to SEQ ID NO: 2;
(c) SEQ ID NO: 3 or a sequence which is fully complementary to SEQ ID NO: 3;
(d) SEQ ID NO: 4 or a sequence which is fully complementary to SEQ ID NO: 4;
(e) SEQ ID NO: 5 or a sequence which is fully complementary to SEQ ID NO: 5;
(f) SEQ ID NO: 6 or a sequence which is fully complementary to SEQ ID NO: 6; and
(g) SEQ ID NO: 7 or a sequence which is fully complementary to SEQ ID NO: 7;
and a second primer is an oligonucleotide selected from the group consisting of:
(h) SEQ ID NO: 8 or a sequence which is fully complementary to SEQ ID NO: 8;
(i) SEQ ID NO: 9 or a sequence which is fully complementary to SEQ ID NO: 9;
(j) SEQ ID NO: 10 or a sequence which is fully complementary to SEQ ID NO: 10
(k) SEQ ID NO: 11 or a sequence which is fully complementary to SEQ ID NO: 11
(l) SEQ ID NO: 12 or a sequence which is fully complementary to SEQ ID NO: 12;
(m)SEQ ID NO: 13 or a sequence which is fully complementary to SEQ ID NO: 13; and
(n) SEQ ID NO: 14 or a sequence which is fully complementary to SEQ ID NO: 14.

4. The general primer pair as in claim 3, wherein the first primer is (a) and the second primer is (h), the primer pair amplifies the Human Papillomavirus genotypes 1a, 2a, 3, 4, 6b, 7, 10, 11, 13, 16, 16r, 18, 26, 27, 28, 30, 31, 32, 33, 34, 36, 35h, 39, 40, 42, 43, 44, 45, 51, 52, 53, 54, 55, 56, 57, 58, 59, 60, 61, 65, 66, 67, 68, 70, 72, 73, 74 and 77.

5. The general primer pair as in claim 3, wherein the first primer is (b) and the second primer is (i), the primer pair amplifies the Human Papillomavirus genotypes 1a, 2a, 3,4, 6b, 7, 10, 11, 13, 16, 16r, 18, 26, 27, 28, 30, 31, 32, 33, 34, 35, 35h, 39, 40, 42, 43, 44, 45, 48, 50, 51, 52, 53, 54, 55, 56, 57, 58, 59, 60, 61, 65, 66, 67, 68, 70, 72, 73, 74and 77.

6. The general primer pair as in claim 3, wherein the first primer is (c) and the second primer is (j), the primer pair amplifies the Human Papillomavirus genotypes 2a, 3, 6b, 7, 10, 11, 13, 16, 16r, 18, 26, 27, 28, 29, 30,31, 32, 33, 34, 35, 35h, 39, 40, 42, 43, 44, 45, 48, 49, 50, 51, 52, 53, 54, 55, 56, 57, 58, 59, 60, 61, 62, 63, 66, 67, 68, 70, 72, 73, 74, 75 and 76.

7. The general primer pair as in claim 3, wherein the first primer is (d) and the second primer is (k), the primer pair amplifies the Human Papillomavirus genotypes 3, 6b, 7, 10, 11, 13, 16, 18, 26, 29, 30, 31, 32, 33, 34, 35, 39, 40, 42, 43, 44, 45, 51, 52, 53, 54, 55, 56, 58, 59, 60, 61, 62, 64, 66, 68, 70, 73, 74, 75, 76 and 77.

8. The general primer pair as in claim 3, wherein the first primer is (e) and the second primer is (j), the primer pair amplifies the Human Papillomavirus genotypes 2a, 3, 6b, 10, 11, 13, 16, 18, 26, 27, 28, 29, 30, 31, 32, 33, 34, 35, 39, 40, 43, 44, 45, 48, 49, 50, 51, 52, 54, 55, 56, 57, 58, 59, 61, 62, 64, 66, 67, 68, 70, 72, 73, 75, 76 and 77.

9. The general primer pair as in claim 3, wherein the first primer is (f) and the second primer is (l), the primer pair amplifies the Human Papillomavirus genotypes 2a, 7, 10, 11, 13, 16, 18, 28, 29, 30, 31, 32, 33, 35, 39, 40, 42, 43, 44, 45, 52, 53, 54, 55, 56, 58, 59, 61, 63, 66, 67, 68, 70, 72, 75, 75 and 77.

10. The general primer pair as in claim 3, wherein the first primer is (g) and the second primer is (m), the primer pair amplifies the Human Papillomavirus genotypes 2a, 3, 6b, 7, 10, 11, 13, 16, 18, 26, 29, 30, 31, 32, 33, 34, 35, 39, 40, 42, 43, 44, 45, 51, 52, 53, 54, 55, 56, 58, 59, 60, 61, 65, 66, 67, 68, 70, 73, 74 and 77.

11. The general primer pair as in claim 3, wherein the first primer is (g) and the second primer is (n), the primer pair amplifies the Human Papillomavirus genotypes 2a, 3, 6b, 7, 10, 11, 13, 16, 18, 26, 29, 30, 31, 32, 33, 34, 35, 39, 40, 42, 43, 44, 45, 51, 52, 53, 54, 55, 56, 58, 59, 60, 61, 65, 66, 67, 68, 70, 73, 74 and 77.

12. The general primer pair as in claim 3, wherein the first primer is (d) and the second primer is (h), the primer pair amplifies the Human Papillomavirus genotypes 3, 6b, 7, 10, 11, 13, 16, 18, 26, 29, 30, 31, 32, 33, 34, 35, 39, 40, 42, 43, 44, 45, 51, 52, 53, 54, 55, 56, 58, 59, 60, 61, 65, 66, 67, 68, 70, 73 and 74.

13. A method of amplifying DNA of Human Papillomavirus genotypes comprising performing a nucleic acid amplification process using a pair of general primers according to claim 3.

14. The method as in claim 13, wherein the pair of general primers are used at concentrations of 20˜50 pM.

15. A method of analyzing a sample for the presence therein of Human Papillomavirus genotypes which comprises the steps of:
(a) amplifying DNA of a Human Papillomavirus in the sample by means of a nucleic acid amplification process using a pair of general primers according to claim 3; and
(b) detecting an amplification product wherein the occurrence of the amplification product indicates presence of Human Papillomavirus genotypes in the sample.

16. The method as in claim 15, wherein the amplification product is labeled.

17. The method as in claim 15, wherein the amplification step is carried out at the general primers concentration of 20˜50 pM.

18. The method as in claim 15, wherein the sample is obtained from a cervical cell.

19. A kit comprising:
(a) a pair of primer according to claim 3;
(b) complex of dNTP (DATP, dGTP, dCTP, and dTTP);
(c) polymerase; and
(d) PCR buffer solution.
